(19) United States
(12) Patent Application Publication
(10) Pub. No.: US 2007/0275949 A1
(43) Pub. Date: Nov. 29, 2007

(54) Benzazepine Derivatives and Methods of Prophylaxis or Treatment of 5Ht2C Receptor Associated Diseases

(75) Inventors: Brian Smith, San Diego, CA (US); Jeffrey A. Schultz, San Diego, CA (US); Charles A. Gilson III, San Diego, CA (US); Scott A. Estrada, La Jolla, CA (US)

Correspondence Address: FISH & RICHARDSON P.C., P.O. BOX 1022, MINNEAPOLIS, MN, 55440-1022, US

(73) Assignee: Arena Pharmaceuticals, Inc., San Diego, CA (US)

(21) Appl. No.: 10/573,196

(22) PCT Filed: Oct. 21, 2004
(86) PCT No.: PCT/US04/34914

§ 371 Date: Mar. 29, 2007

Related U.S. Application Data
(60) Provisional application No. 60/513,894, filed on Oct. 22, 2003.

(51) Int. Cl. A61K 31/55; A61P 43/00; C07D 223/16

(52) U.S. Cl. 514/217.02; 540/595

(57) ABSTRACT

The present invention relates to substituted-2,3,4,5-tetrahydro-3-benzazepine derivatives that are modulators of the 5HT_{2C} receptor. Accordingly, compounds of the present invention are useful for the prophylaxis treatment of 5HT_{2C} receptor associated diseases, conditions or disorders, such as, obesity and related disorders.

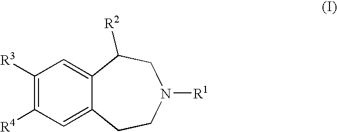

### FIELD OF THE INVENTION

The present invention relates to substituted-2,3,4,5-tetrahydro-3-benzazepine derivatives that are modulators of the 5HT_{2C} receptor. Accordingly, compounds of the present invention are useful for treatment of 5HT_{2C} receptor-associated diseases, conditions or disorders, such as, obesity and related disorders.

### BACKGROUND OF THE INVENTION

Obesity is a life-threatening disorder in which there is an increased risk of morbidity and mortality arising from concomitant diseases such as, but not limited to type II diabetes, hypertension, stroke, certain forms of cancers and gallbladder disease.

Obesity has become a major healthcare issue in the Western World and increasingly in some third world countries. The increase in the number of obese people is due largely to the increasing preference for high fat content foods but also, and this can be a more important factor, the decrease in activity in most people's lives. In the last 10 years there has been a 30% increase in the incidence of obesity in the USA and that about 30% of the population of the USA is now considered obese. In spite of the growing awareness of the health concerns linked to obesity the percentage of individuals that are overweight or obese continue to increase. In fact, the percentage of children and adolescents who are defined as overweight has more than doubled since the early 1970s and about 13 percent of children and adolescents are now seriously overweight. The most significant concern, from a public health perspective, is that children who are overweight grow up to be overweight or obese adults, and accordingly are at greater risk for major health problems. Therefore, it appears that the number of individuals that are overweight or obese will continue to increase.

Whether someone is classified as overweight or obese is generally determined on the basis of his or her body mass index (BMI) which is calculated by dividing their body weight (kilograms—Kg) by their height squared (meters squared—m²). Thus, the units for BMI are Kg/m². The BMI is more highly correlated with body fat than any other indicator of height and weight. A person is considered overweight when they have a BMI in the range of 25-30 kg/m². Whereas a person with a BMI over 30 kg/m² is classified as obese and obesity is further divided into three classes, Class I (BMI of about 30 to about 34.9 kg/m²), Class II (BMI of about 35 to 39.9 kg/m²) and Class III (about 40 kg/m² or greater); see TABLE I below for complete classifications.

TABLE I
CLASSIFICATION OF WEIGHT BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases for an individual there is an increased risk of morbidity and mortality relative to an individual with normal BMI. Accordingly, overweight and obese individuals (BMI of about 25 kg/m² and above) are at increased risk for physical ailments such as, but not limited to, high blood pressure, cardiovascular disease (particularly hypertension), high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), diseases of reproduction (such as sexual dysfunction, both male and female, including male erectile dysfunction), bladder control problems (such as stress incontinence), uric acid nephrolithiasis, psychological disorders (such as depression, eating disorders, distorted body image, and low self esteem). Research has shown that even a modest reduction in body weight can correspond to a significant reduction in the risk of developing other ailments, such as, but not limited to, coronary heart disease.

As mentioned above, obesity increases the risk of developing cardiovascular diseases. Coronary insufficiency, atheromatous disease, and cardiac insufficiency are at the forefront of the cardiovascular complications induced by obesity. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of obesity [Perry, I. J., et al. BMJ 310, 560-564 (1995)]. It is estimated that if the entire population had an ideal weight, the risk of coronary insufficiency would decrease by 25% and the risk of cardiac insufficiency and of cerebral vascular accidents by 35%.

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina and increases the risk of cataracts and glaucoma Finally, diabetes is associated with nerve damage, especially in the legs and feet, which interferes with the ability to sense pain and contributes to serious infections. Taken together, diabetes complications are one of the nation's leading causes of death.

The first line of treatment for individuals that are overweight or obese is to offer diet and life style advice, such as, reducing the fat content of their diet and increasing their physical activity. However many patients find these difficult to maintain and need additional help from drug therapy to sustain results from these efforts.

Most currently marketed products have been unsuccessful as treatments for obesity owing to a lack of efficacy or unacceptable side-effect profiles. The most successful drug so far was the indirectly acting 5-hydroxytryptamine (5-HT) agonist d-fenfluramine (Redux™) but reports of cardiac valve defects in up to one third of the patient population led to its withdrawal by the FDA in 1998.

In addition, two drugs have recently been launched in the USA and Europe: Orlistat (Xenical™), a drug that prevents absorption of fat by the inhibition of pancreatic lipase, and Sibutraine (Reductil™), a 5-HT/noradrenaline re-uptake inhibitor. However, side effects associated with these products may limit their long-term utility. Treatment with Xenical™ is reported to induce gastrointestinal distress in some patients, while Sibutramine has been associated with raised blood pressure in some patients.

Serotonin (5-HT) neurotransmission plays an important role in numerous physiological processes both in health and in psychiatric disorders. 5-HT has been implicated in the regulation of feeding behavior for some time. 5-HT works by inducing a feeling of fullness or satiety so eating stops earlier and fewer calories are consumed. It has been shown that a stimulatory action of 5-HT on the 5HT_{2C} receptor plays an important role in the control of eating and in the anti-obesity effect of d-fenfluramine. As the 5HT_{2C} receptor is expressed in high density in the brain (notably in the limbic structures, extrapyramidal pathways, thalamus and hypothalamus i.e. PVN and DMH, and predominantly in the choroid plexus) and is expressed in low density or is absent in peripheral tissues, a selective 5HT_{2C} receptor agonist can be an effective and safe anti-obesity agent. Also, 5HT_{2C} knockout mice are overweight with cognitive impairment and susceptibility to seizure thus establishing the clear use for a 5HT_{2C} receptor agonist in 5HT_{2C} receptor associated diseases or disorders.

The 5HT_{2C} receptor plays a role in obsessive compulsive disorder, some forms of depression, and epilepsy. Accordingly, 5HT_{2C} receptor agonists can have anti-panic properties, and properties useful for the treatment of sexual dysfunction. In addition, 5HT_{2C} receptor agonists are useful for the treatment of psychiatric symptoms and behaviors in individuals with eating disorders such as, but not limited to, anorexia nervosa and bulimia nervosa. Individuals with anorexia nervosa often demonstrate social isolation. Anorexic individuals often present symptoms of being depressed, anxious, obsession, perfectionistic traits, and rigid cognitive styles as well as sexual disinterest. Other eating disorders include, anorexia nervosa, bulimia nervosa, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. What the individual is doing with regard to food and weight is neither normal nor healthy.

In addition, the 5HT_{2C} receptor is also involved in other diseases, conditions and disorders; such as Alzheimer Disease (AD). Therapeutic agents currently prescribed for Alzheimer's disease (AD) are cholinomimetic agents that act by inhibiting the enzyme acetylcholinesterase. The resulting effect is increased levels of acetylcholine, which modestly improves neuronal function and cognition in patients with AD. Although, dysfunction of cholinergic brain neurons is an early manifestation of AD, attempts to slow the progression of the disease with these agents have had only modest success, perhaps because the doses that can be administered are limited by peripheral cholinergic side effects, such as tremors, nausea, vomiting, and dry mouth. In addition, as AD progresses, these agents tend to lose their effectiveness due to continued cholinergic neuronal loss.

Therefore; there is a need for agents that have beneficial effects in AD, particularly in alleviating symptoms by improving cognition and slowing or inhibiting disease progression, without the side effects observed with current therapies. Therefore, serotonin 5HT_{2C} receptors, which are exclusively expressed in brain, are attractive targets.

A major feature of AD is the formation of senile plaques made of amyloid deposits in a selected area of the brain; New therapies should focus on prevention of the production of these senile plaques. An amyloid deposit composed mainly of beta-amyloid peptide (Aβ) occupies the plaque center. Aβ is a peptide of 40 to 43 residues derived from a larger amyloid precursor protein, APP [Selkoe D J, et al. Ann Rev Neurosci, 1994, 17:489-517]. APP is a ubiquitous transmembrane glycoprotein that is present at high levels in brain cells. APP also exists as secreted forms. By cleavage in the Aβ region of APP, the long N-terminal fragment (secreted APP, APPs) is secreted into the extracellular space. The rate of Aβ production appears to be inversely coupled to rate APPs secretion. In several cell cultures, APPs secretion was accompanied by reductions in secreted Aβ [Buxbaum J D, et al. Proc Nat Acad Sci, 1993, 90:9195-9198; Gabuzda D, et al. J Neurochem, 1993, 61:2326-2329; Hung A Y, et al. J Biol Chem, 1993, 268:22959-22962; and Wolf B A, et al. J Biol Chem, 1995, 270:4916-4922], suggesting that stimulated secretory processing of APP into secreted APPs is associated with reduced formation of potentially amyloidogenic derivatives, or plaques.

APPs is found in plasma and cerebrospinal fluid [Ghiso J, et al. Biochem Biophys Res Comm, 1989, 163:430-437; and Podlisny M B, et al. Biochem Biophys Res Commun, 1990, 167:1094-1101]. Considering the abundance of both membrane-bound APP and APPs, they are likely to have significant biological functions. Current knowledge about APP functions indicates APP is critically required for the maintenance of neuronal and synaptic structure and function. Membrane-bound APP has been suggested to have a receptor-like structure [Kang J, et al. Nature, 1987, 325:733-736], with the cytoplasmic domain capable of complexing with a GTP-binding protein [Nishimoto I., et al. Nature, 1993, 362:75-79]. Membrane-embedded full-length APP might also have a cell adhesion function [Qiu W., et al. J Neurosci, 1995, 15:2157-2167].

APPs has been shown to be neurotrophic and neuroprotective in vitro [Mattson M P, et al. Neuron, 1993, 10:243-254; and Qiu W., et al. J Neurosci, 1995, 15:2157-2167]. Other proposed functions for APPs include the regulation of blood coagulation [Cole GM, et al. Biochem Biophlys Res Commun, 1990, 170:288-295; Smith R P, et al. Science, 1990, 248:1126-1128; and Van Nostrand et al. Science, 1990, 248:745-748], wound-healing [Cunningham J M, et al. Histochemistry, 1991, 95:513-517], extracellular protease activity [Oltersdorf T, et al. Nature (London), 1989, 341:144-147; and Van Nostrand W E, et al. Nature, 1989, 341:546-548], neurite extension [Jin L., et al. J Neurosci, 1994, 14:5461-5470; and Robakis N K, et al. in Molecular Biology of Alzheimer's Disease. (T. Miyatake, D. J. Selkoe and Y. Ihara, ed.), 1990, pp. 179-188, Elsevier Science Publishers B. V., Amsterdam], cell adhesiveness [Schubert D, et al. Neuron, 1989, 3:689-694], cell growth, [Bhasin R., et al. Proc Natl Acad Sci USA, 1991, 88:10307-10311; and Saitoh T., Cell, 1989, 58:615-622], and differentiation [Araki W., et al. Biochem Biophys Res Commun,1991, 181:265-271; Milward E A, et al. Neuron, 1991, 9:129-137; and Yamamoto K, et al. J Neurobiol, 1994, 25:585-594].

The non-selective serotonin 5HT_{2C} agonist dexnorfenfluramine (DEXNOR) stimulated amyloid precursor protein (APPs) secretion in guinea pigs while reducing levels of Aβ production in vivo following repeat administration [Arjona A, et al. “Effect of a 5HT_{2C} serotonin agonist, dexnorfenfluramnine, on amyloid precursor protein metabolism in guinea pigs,” Brain Res, 2002, 951:135-140]. Guinea pigs were chosen because guinea pig and human APP exhibit 98% sequence homology [Beck M, et al. Biochem Biophys Acta, 1997, 1351:17-21], the proteins are processed similarly [Beck M., et al. Neuroscience, 1999, 95:243-254], and the Aβ peptide sequences are identical [Johnstone E M, et al. Brain Res Mol Brain Res, 1991, 10:299-305]. Although DEXNOR is non-selective, the observed effects were attenuated by a selective serotonin 5HT_{2C} antagonist, while a selective serotonin HT_{2A} antagonist did not reverse the DEXNOR effects, indicating the serotonin 5HT_{2C} receptors are the most relevant target for this effect.

In addition, 5-HT stimulates APPs ectodomain secretion via the serotonin 5HT_{2A} and 5HT_{2C} receptors [Nitsch R M, et al. J Biol Chem, 1996, 271(8):4188-4194]. In this study, researchers stimulated 3T3 fibroblasts with serotonin (5-HT), which were stably expressing serotonin 5HT_{2A} or 5HT_{2C} receptors. 5-HT increased APPs secretion in a dose-dependent manner in both cell lines. Maximal stimulation of APPs secretion peaked at about 4-fold. Selective serotonin 5HT_{2A} and 5HT_{2C} antagonists blocked the effects in each cell line.

A serotonin 5HT_{2C} receptor agonist can be effective for treating AD and preventing senile plaques. Support for this claim comes from the fact that Aβ is known to be neurotoxic and a key component in senile plaques involved in AD, APPs secretion and Aβ levels seem to be inversely related, and serotonin 5HT_{2C} agonists increase levels of APPs in vitro in cell lines stably expressing serotonin 5HT_{2C} receptors while in vivo serotonin 5HT_{2C} agonists increase levels of APPs and decrease levels of Aβ as measured in cerebral spinal fluid of guinea pigs.

Evidence exists supporting the use of a compound of the present invention with agonist activity at the serotonin 5HT_{2C} receptor for the treatment of AD. The compound of the invention can be used alone or in combination with another agent or agents (such as but not limited to AChE inhibitors) that are typically prescribed for AD.

Another disease, disorder or condition that can is associated with the function of the 5HT_{2C} receptor is erectile dysfunction (ED). Erectile dysfunction is the inability to achieve or maintain an erection sufficiently rigid for intercourse, ejaculation, or both. An estimated 20-30 million men in the United States have this condition at some time in their lives. The prevalence of the condition increases with age. Five percent of men 40 years of age report ED. This rate increases to between 15% and 25% by the age of 65, and to 55% in men over the age of 75 years.

Erectile dysfunction can result from a number of distinct problems. These include loss of desire or libido, the inability to maintain an erection, premature ejaculation, lack of emission, and inability to achieve an orgasm. Frequently, more than one of these problems presents themselves simultaneously. The conditions may be secondary to other disease states (typically chronic conditions), the result of specific disorders of the urogenital system or endocrine system, secondary to treatment with pharmacological agents (e.g. antihypertensive drugs, antidepressant drugs, antipsychotic drugs, etc.) or the result of psychiatric problems. Erectile dysfunction, when organic, is primarily due to vascular irregularities associated with atherosclerosis, diabetes, and hypertension.

There is evidence for use of a serotonin 5HT_{2C} agonist for the treatment of sexual dysfunction in males and females. The serotonin 5HT_{2C} receptor is involved with the processing and integration of sensory information, regulation of central monoaminergic systems, and modulation of neuroendocrine responses, anxiety, feeding behavior, and cerebrospinal fluid production [Tecott, L. H., et al. Nature 374: 542-546 (1995)]. In addition, the serotonin 5HT_{2C} receptor has been implicated in the mediation of penile erections in rats, monkeys, and humans.

The exact mechanism by which 5HT_{2C} receptors mediate penile erections remains unknown. However, there is good evidence, indirect and direct, supporting the role of serotonin 5HT_{2C} receptors in the mediation of penile erections. Anatomical studies have shown that the penis receives autonomic innervation from sympathetic and parasympathetic nuclei located in the spinal cord [Pescatori E S, et al. J Urol 1993; 149: 627-32]. In agreement, experimental and clinical data support that penile erections are controlled by a spinal reflex. A closer analysis showed that activation of 5HT2 spinal receptors facilitated pudendal reflex in anesthetized cats [Danuser H and Thor K B, Br J Pharmacol 1996; 118: 150-4]. Accordingly, stimulation of 5HT_{2C} receptors has been shown to be proerectile [Milan M J, et al. European Journal of Pharmacology 1997; 325], and 5HT_{2C} receptors have been described on proerectile spinal parasympathetic neurons [Bancila M et al. Neuroscience 1999; 92: 1523-37].

Indirect evidence comes from the research and reports of the side effects induced by the use of selective serotonin reuptake inhibitors (SSRIs). SSRIs have demonstrated antagonist action at the serotonin 5HT_{2C} receptors [Jenck et al. European Jounral of Pharmacology 231: 223-229 (1993); Lightlowler et al. European Journal of Pharmacology 296: 137-43 (1996); and Palvimaki, E., et al. Psychopharmacology 126: 234-240 (1996)). Among the most derogatory side effects of SSRIs noted in humans is increased difficulty in attaining penile erection. Although SSRIs have a rich pharmacological profile, it is believed that the antagonist effects of SSRIs at the 5HT_{2C} receptors could be implicated in the inhibition of penile erections [Palvimaki, E., et al. Psychopharmacology 126: 234-240 (1996)].

Further evidence comes from studies with a variety compounds with known agonist activity for the serotonin 5HT_{2C} receptor. Pharmacologic studies with rats and rhesus monkeys provide direct evidence of the proerectile properties of agonist of the serotonin 5-HT_{2C} receptor [Millan M J, et al. European Journal of Pharmacology 1997; 325; and Pomerantz, et al. European Journal of Pharmacology 243:227-34 (1993)]. These pro-erectile effects were unaffected by antagonists for the serotonin 5HT_{2A} and 5HT_{2B} receptors, respectively; Antagonists of the serotonin 5HT_{2C} receptors attenuated the proerectile effects of the 5-HT_{2C} agonists. The inhibition action corresponded to each antagonist's affinity for the 5-HT_{2C} receptors. In addition, agonists of the serotonin 5HT_{2A} and 5HT_{2B} receptors did not elicit penile erections.

It is widely believed that HDL is a “protective” lipoprotein (Vega et al., Current Opinion in Lipidology, 1996, 7, 209-216) and that increasing plasma levels of HDL may offer a direct protection against the development of atherosclerosis. Numerous studies have demonstrated that both the risk of coronary heart disease (CHD) in humans and the severity of experimental atherosclerosis in animals are inversely correlated with serum HDL-cholesterol (HDL-C) concentrations (Russ et al., Am. J. Med., 1951, 11, 480-483; Gofman et al., Circulation, 1966, 34, 679-697; Miller et al., Lancet, 1975, 1, 16-19; Gordon et al., Circulation, 1989, 79, 8-15; Stampfer et al., N. Engl. J Med., 1991, 325, 373-381; Badimon et al., Lab. Invest., 1989, 60, 455461). Atherosclerosis is the process of the accumulation of cholesterol within the arterial wall which results in the occlusion, or stenosis, of coronary and cerebral arterial vessels and subsequent myocardial infarction and stroke. Angiographic studies have shown that elevated levels of some HDL particles in humans appear to be correlated to a decreased number of sites of stenosis in the coronary arteries of humans (Miller et al., Br. Med. J., 1981, 282, 1741-1744).

There are several mechanisms by which HDL may protect against the progression of atherosclerosis. Studies in vitro have shown that HDL is capable of removing cholesterol from cells (Picardo et al., Arteriosclerosis, 1986, 6, 434-441). Data of this nature suggest that one antiatherogenic property of HDL may lie in its ability to deplete tissue of excess free cholesterol and eventually lead to the delivery of this cholesterol to the liver (Glomset, J. Lipid Res., 1968, 9, 155-167). This has been supported by experiments showing efficient transfer of cholesterol from HDL to the liver (Glass et al., J. Biol. Chem., 1983, 258, 7161-7167; McKinnon et al., J. Biol. Chem., 1986, 26, 2548-2552). In addition, HDL may serve as a reservoir in the circulation for apoproteins necessary for the rapid metabolism of triglyceride-rich lipoproteins (Grow and Fried, J. Biol. Chem., 1978, 253, 1834-1841; Lagocki and Scanu, J. Biol Chem., 1980, 255, 3701-3706; Schaefer et al., J. Lipid Res., 1982, 23, 1259-1273).

Generally, the total cholesterol/HDL-cholesterol (i.e., TC/HDL) ratio represents a useful predictor as to the risk of an individual in developing a more serious condition, such as a HDL-related condition. The classification of plasma lipid levels is shown in Table A:

TABLE A
CLASSIFICATION OF PLASMA LIPID LEVELS

| | | | |
|---|---|---|---|
| TOTAL | <200 | mg/dl | Desirable |
| CHOLESTEROL | 200-239 | mg/dl | Borderline High |
| | >240 | mg/dl | High |
| TOTAL | <40 | mg/dl | Low (Men) |
| CHOLESTEROL | <50 | mg/dl | Low (Women) |
| | <200 | mg/dl | Desirable |

From: 2001 National Cholesterol Education Program Guidelines

Accordingly, the recommended total cholesterol/HDL-C (i.e., TC/HDL) ratio indicates that a ratio of less than or equal to 3.5 is ideal and a ratio of greater than 4.5 is considered an increased “at risk.” The value of determining the TC/HDL ratio is clearly evident in the circumstance where an individual presents with “normal” LDL and total cholesterol but possesses low HDL-cholesterol. Based on LDL and total cholesterol the individual may not qualify for treatment, however, factor in the HDL-cholesterol level then a more accurate risk assessment may be obtained. Thus, if the individual's level of HDL-cholesterol is such that the ratio is greater than 4.5 then therapeutic or prophylactic intervention may be warranted. A physician or care provider may determine the need of prophylaxis or treatment based on a TC/HDL ratio; for example, a TC/HDL ratio of 2.5 or greater, 3.0 or greater, 3.5 or greater, 4.0 or greater, 4.5 or greater, 5.0 or greater, or a TC/HDL ratio of 5.5 or greater.

Accordingly, agents that increase HDL-C levels or reduce total cholesterol/HDL-C ratios would be of utility as antiatherosclerotic agents, and particularly useful in the prophylaxis or treatment of coronary heart disease, ischemic cerebrovascular disease, peripheral vascular disease and dyslipoproteinimias.

In summary, the 5HT_{2C} receptor is a validated and well-accepted receptor target for the prophylaxis and/or treatment of 5HT_{2C} mediated receptor diseases and disorders, such as, obesity, eating disorders, psychiatric disorders, Alzheimer Disease, sexual dysfunction and disorders related thereto. It can be seen that there exists a need for selective 5HT_{2C} receptor agonists that can safely address these needs. The present invention is directed to these, as well as other, important ends.

### SUMMARY OF THE INVENTION

The present invention provides 3-benzazepine compounds that can modulate the 5HT_{2C} receptor, and in some embodiments, are agonists of the receptor.

The present invention provides a compound of Formula (I): 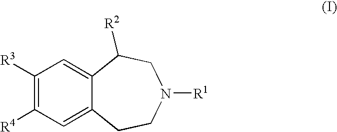
or pharmaceutically acceptable salt thereof, wherein:

R¹ is H or C₁-C₈ alkyl;

R² is C₁-C₈ alkyl;

R³ is H, aryl, arylalkyl-O—, arylalkyl-N(R⁵)—, aryl-N(R⁵)—, or heteroaryl, wherein said aryl is optionally substituted with up to two substituents selected from C₁₋₈ alkyl, halogen, perhaloalkyl, and alkoxy;

R⁴ is H, arylalkyl-O—, alkoxy, or aryloxy; and

R⁵ is H, C₁-C₈ alkyl, aryl, C₁₋₈ alkenyl, heteroaryl, arylalkyl, heteroarylalkyl, perhaloalkyl, or allyl.

In some embodiments, compounds of the present invention are of Formula (I) wherein:

R¹ is H or C₁-C₈ alkyl;

R² is C₁-C₈ alkyl;

R³ is H, aryl, arylalkyl-O—, arylalkyl-N(R⁵)—, or aryl-N(R⁵)—, wherein said aryl is optionally substituted with up to two substituents selected from C₁₋₈ alkyl, halogen, perhaloalkyl, and alkoxy;

R⁴ is H or aryloxy; and

R⁵ is H, C_{1-C}₈ alkyl, aryl, C₁₋₈ alkenyl, heteroaryl, arylalkyl, heteroarylalkyl, perhaloalkyl, or allyl.

The present invention further provides a composition comprising a compound of Formula (I) and a pharmaceutically acceptable carrier.

The present invention frther provides a method of modulating a 5HT_{2C} receptor comprising contacting the receptor with a compound of Formula (I).

The present invention furter provides a method of treating disorders of the central nervous system, damage to the central nervous system, cardiovascular disorders, gastrointestinal disorders, diabetes insipidus, sleep apnea or HDL-related condition comprising administering to a patient in need of the treating a therapeutically effective amount of a compound of Formula (I).

The present invention further provides a method of decreasing food intake of a mammal comprising administering to the mammal a therapeutically effective amount of a compound of Formula (I).

The present invention further provides a method of inducing satiety in a mammal comprising administering to the mammal a therapeutically effective amount of a compound of Formula (I).

The present invention further provides a method of controlling weight gain of a mammal comprising administering to the mammal a therapeutically effective amount of a compound of Formula (I).

The present invention further provides a method of treating obesity comprising administering to a patient in need of such treating a therapeutically effective amount of a compound of Formula (I).

The present invention further provides a compound, as described herein, for use in a method of treatment of the human or animal body by therapy.

The present invention further provides a compound of the present invention for manufacture of a medicament for use in treating disorders of the central nervous system; damage to the central nervous system; cardiovascular disorders; gastrointestinal disorders; diabetes insipidus; sleep apnea or HDL-related condition.

In some embodiments, disorders of the central nervous system include, for example, depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension. In some embodiments, sexual dysfunction is male erectile dysfunction.

### DETAILED DESCRIPTION OF EMBODIMENTS OF THE INVENTION

The present invention provides, inter alia, a compound of Formula (I): 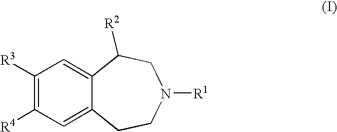
or pharmaceutically acceptable salt thereof, wherein:

R¹ is H or C_{1-C}₈ alkyl;

R² is C₁-C₈ allyl;

R³ is H, aryl, arylalkyl-O—, arylalkyl-N(R⁵)—, aryl-N(R⁵)—, or heteroaryl, wherein said aryl is optionally substituted with up to two substituents selected from C₁₋₈ alkyl, halogen, perhaloalkyl, and alkoxy;

R⁴ is H, arylalkyl-O—, alkoxy, or aryloxy; and

R⁵ is H, C₁-C₈ alkyl, aryl, C₁₋₈ alkenyl, heteroaryl, arylalkyl, heteroarylalkyl, perhaloalkyl, or allyl.

In some embodiments, compounds of the present invention are of Formula (I) wherein:

R¹ is H or C₁-C₈ alkyl;

R² is C₁-C₈ alkyl;

R³ is H, aryl, arylalkyl-O—, arylalkyl-N(R⁵)—, or aryl-No⁵)—, wherein said aryl is optionally substituted with up to two substituents selected from C₁₋₈-alkyl, halogen, perhaloalkyl, and alkoxy;

R⁴ is H or aryloxy; and

R⁵ is H, C₁-C₈ alkyl, aryl, C₁₋₈ alkenyl, heteroaryl, arylalkyl, heteroarylalkyl, perhaloalkyl, or allyl.

In some embodiments, at least one of R³ and R⁴ is other than H.

In some embodiments, when R³ is arylalkyl-N(R⁵)— or aryl-N(R⁵)— and R⁴ is H, then R¹ is H.

In some embodiments, when R¹ is H, R² is CH₃ and R³ is 2-chlorophenyl, then R⁴ is other than H.

In some embodiments, when R¹ is H, R² is CH₃ and R³ is 2-thienyl, then R⁴ is other than methoxy.

In some embodiments, when R⁴ is alkoxy, then R³ is other than H.

In some embodiments: R¹ is H or C₁-C₈ alkyl; R² is C₁-C₈ alkyl; R³ is arylalkyl-O—, arylalkyl-N(R⁵)—, or aryl-N(R⁵)—; R⁴ is H; and R⁵ is H, C₁-C₈ alkyl, aryl, C₁₋₈ alkenyl, heteroaryl, arylalkyl, heteroarylalkyl, perhaloalkyl, or allyl.

In some embodiments: R¹ is H or C₁-C₈ alkyl; R² is C₁-C₈ alkyl; R³ is arylalkyl-O—, arylalkyl-N(R⁵)—, or aryl-N(R⁵)—; R⁴ is H; and R⁵ is H, C₁-C₈ alkyl, or aryl.

In some embodiments: R¹ is H or C₁-C₈ alkyl; R² is C₁-C₈ alkyl; R³ is arylalkyl-O—; and R⁴ is H.

In some embodiments: R¹ is H or C₁-C₈ alkyl; R² is C₁-C₈ alkyl; R³ is H; and R⁴ is arylalkyl-O—.

In some embodiments: R¹ is H; R² is C₁-C₄ alkyl; R³ is arylalkyl-O—; and R⁴ is H.

In some embodiments: R¹ is H; R² is C₁-C₈ alkyl; R³ is arylalkyl-N(R⁵)—; R⁴ is H; and R⁵ is H, C₁-C₈ alkyl, or aryl.

In some embodiments: R¹ is H; R² is methyl; R³ is phenyl, phenylalkyl-O—, phenylalkyl-N(R⁵)—, or phenyl-N(R⁵)—; R⁴ is H; and R⁵ is H.

In some embodiments: R¹ is H; R² is methyl; R³ is H; and R⁴ is phenylalkyl-O—.

In some embodiments: R¹ is H; R² is methyl; R³ is phenyl optionally substituted with up to two halogens, or R³ is pyridinyl; and R⁴ is H or alkoxy.

In some embodiments: R¹ is H; R² is methyl; R³ is phenyl optionally substituted with up to two fluorine atoms; and R⁴ is H or methoxy.

In some embodiments: R¹ is H; R² is methyl; R³ is pyridinyl; and R⁴ is H.

In some embodiments, the compound of Formula (I) is selected from:

a) 7-benzloxy-1-methyl-2,3,4,5-tetrahydro-1H-benzo[d]azepine;

b) 1-methyl-7-1 -phenyl-ethoxy)-2,3,4,5-tetaahydro-1H-benzo[d]azepine;

c) 1-methyl-7-phenethyloxy-2,3,4,5-tetrahydro-1H-benzo[d]azepine;

d) 1-methyl-7-3-phenyl-propoxy)-2,3,4,5-tetrahydro-1H-benzo[d]azepine;

e) benzyl-(5-methyl-2,3,4,5-tetrahydro-1H-benzo[d]azepin-7-yl)-amine;

f) (5-methyl-2,3,4,5-tetrahydro-1H-benzo[d]azepin-7-yl)-(1-phenyl-ethyl)-amine;

g) benzyl-methyl(5-methyl-2,3,4,5-tetrahydro-1H-benzo[d]azepin-7-yl)-amine;

h) (5-methyl-2,3,4,5-tetrahydro-1H-benzo[d]azepin-7-yl)-phenethyl-amine;

i) (5-methyl-2,3,4,5-tetrahydro-1H-benzo[d]azepin-7-yl)-(3-phenyl-propyl)-amine;

j) (5-methyl-2,3,4,5-tetrahydro-1H-benzo[d]azepin-7-yl)-phenyl-amine; and

k) 1-methyl-8-phenyl-2,3,4,5-tetrahydro-1H-benzo[d]azepine;

or pharmaceutically acceptable salt thereof.

In some embodiments, the compound of Formula (I) is selected from:

a) 8-Benzyloxy-1-methyl-2,3,4,5-tetrahydro-1H-benzo[d]azepine;

b) 7-Benzyloxy-l-methyl-2,3,4,5-tetrahydro-1H-benzo[d]azepine;

c) 1-Methyl-8-phenyl-2,3,4,5-tetrahydro-1H-benzo[d]azepine;

d) 7-Methoxy-1-methyl-8-phenyl-2,3,4,5-tetrahydro-1H-benzo[d]azepine;

e) 8-(2-Fluoro-phenyl)-1-methyl-2,3,4,5-tetrahydro-1H-benzo[d]azepine;

f) 8-(3-Fluoro-phenyl)-1-methyl-2,3,4,5-tetrahydro-1H-benzo[d]azepine;

g) 8-(4-Fluoro-phenyl)-1-methyl-2,3,4,5-tetrahydro-1H-benzo[d]azepine;

h) 8-(2,6-Difluoro-pbenyl)-1-methyl-2,3,4,5-tetrahydro-1H-benzo[d]azepine;

i) 8-(2,3-Difluoro-phenyl)-1-methyl-2,3,4,5-tetrahydro-1H-benzo[d]azepine;

j) 8-(2,5-Difluoro-phenyl)-1-methyl-2,3,4,5-tetrahydro-1H-benzo[d]azepine;

k) 1-Methyl-8-pyridin-3-yl-2,3,4,5-tetrahydro-1H-benzo[d]azepine; and

l) 1-Methyl-8-pyridin-2-yl-2,3,4,5-tetrahydro-1H-benzo[d]azepine;

or pharmaceutically acceptable salt thereof.

In some embodiments, the compound of the invention has Formula (Ia). 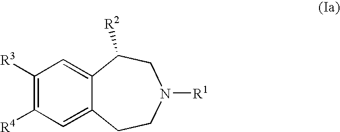
or pharmaceutically acceptable salt thereof.

In other embodiments, the compound of the invention has Formula (Ib). 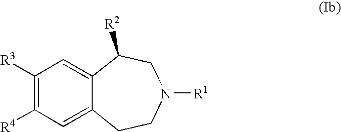
or pharmaceutically acceptable salt thereof.

It is appreciated that certain features of the invention, which are, for clarity, described in the context of separate embodiments, may also be provided in combination in a single embodiment. Conversely, various features of the invention which are, for brevity, descnbed in the context of a single embodiment, may also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like.

As used herein, “alkynyl” refers to an alkyl group having one or more triple carbon-carbon bonds. Example alkynyl groups include ethynyl, propynyl, butynyl, pentynyl, and the like.

As used herein, “haloalkyl” refers to an alkyl group having one or more halogen substituents. Example haloalkyl groups include CF₃, C₂F₅, CHF₂, CCl₃; CHCl₂, C₂Cl₅, and the like. An alkyl group in which all of the hydrogen atoms are replaced with halogen atoms can be referred to as “perhaloalkyl.” Examples perhaloalkyl groups include CF₃ and C₂F₅.

As used herein, “aryl” refers to monocyclic or polycyclic aromatic hydrocarbons such as, for example, phenyl, naphthyl, anthracenyl, phenanthrenyl, indanyl, indenyl, and the like. In some embodiments, aryl groups have from 6 to about 18 carbon atoms.

As used herein, “cycloalkyl” refers to non-aromatic cyclic hydrocarbons, including cyclized alkyl, alkenyl, and alkynyl groups. Cycloalkyl group can include bi- or poly-cyclic ring systems and can optionally contain unsaturations. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like. Cycloalkyl groups can have from about 3 to about 20, 3 to about 12, or 3 to about 7 carbon atoms.

As used herein, “heteroaryl” groups are monocyclic and polycyclic aromatic hydrocarbons that have at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrrolyl, oxazolyl, benzofuryl, benzothienyl, benzthiazolyl, isoxazolyl, pyrazolyl, triazolyl, tefazolyl indazolyl, 1,2,4thiadiazolyl, isothiazolyl, benzothienyl, purinyl, carbazolyl, benzimidazolyl, 2,3-dihydrobenzofuranyl, 2,3-dihydrobenzothienyl, 2,3-dihydrobenzothienyl-S-oxide, 2,3-dihydrobenzothienyl-S-dioxide, benzoxazolin-2-on-yl, indoliiyl, benzodioxolanyl, benzodioxane, and the like. In some embodiments, heteroaryl groups can have from 1 to about 20 carbon atoms, and in further embodiments from about 3 to about 20 carbon atoms. In some embodiments, heteroaryl groups have 1 to about 4, 1 to about 3, or 1 to 2 heteroatoms.

As used herein, “heterocycloalkyl” refers to a non-aromatic hydrocarbon including cyclized alkyl, alkenyl, and alkynyl groups where one or more of the ring-forming carbon atoms is replaced by a heteroatom such as an O, N, or S atom. Also included in the definition of heterocycloalkyl are moieties that have one or more aromatic rings fused (i.e., having a bond in common with) to the nonaromatic heterocyclic ring, for example phthalimidyl, naphthalimidyl pyromellitic diimidyl, phthalanyl, and benzo derivatives of saturated heterocycles such as indolene and isoindolene groups.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like. “Haloalkoxy” refers to an —O-haloalkyl group.

As used herein, “aryloxy” refers to an —O-aryl group. An example aryloxy group is phenoxy.

As used herein, “aralkyl” or “arylalkyl” refers to an alkyl moiety substituted by an aryl group. Example aralkyl groups include phenylalkyl groups such as benzyl, phenethyl (1-phenylethyl or 2-phenylethyl), phenpropyl, naphthylmethyl, and thelike. In some embodiments, aralkyl groups have from 7 to 11 carbon atoms.

As used herein, “heteroarylalkyl” refers to an alkyl moiety substituted by a heteroaryl moiety.

As used herein, “substituted” indicates that at least one hydrogen atom of a chemical group is replaced by a non-hydrogen moiety. When a chemical group herein is “substituted” it may have up to the full valance of substitution, provided the resulting compound is a stable compound or stable structure; for example, a methyl group may be substituted by 1, 2, or 3 substituents, a methylene group may be substituted by 1 or 2 substituents, a phenyl group may be substituted by 1, 2, 3, 4, or 5 substituents, and the like.

As used herein “stable compound” or “stable structure” refers to a compound that is sufficiently robust to survive isolation to a useful degree of purity from a reaction mixture, and preferably capable of formulation into an efficacious therapeutic agent; The present invention is directed only to stable compounds.

The compounds described herein can be asymmetric (e.g., having, one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g:, dinitrobenzoylphenylglycine). Suitable elution solvent composition can be determined by one skilled in the art.

Compounds of the invention can also include tautomeric forms, such as keto-enol tautomers. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amnino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, fornate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward D. Roche, American Pharmaceutical Association and Pergamon Press, 1987, both of which are hereby incorporated by reference in their entirety.

Synthesis

Compounds of the invention, including salts, hydrates, and solvates thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Preparation of compounds of the invention can involve the protection and deprotection of various chemical groups. The need for protection and deprotection, and the selection of appropriate protecting groups can be readily determined by one skilled in the art. The chemistry of protecting groups can be found, for example, in T. W. Green and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Compounds of the invention can be generally prepared by the methods illustrated below, according to Schemes I through V, Infra. One representative synthesis is set forth below in Scheme I, for when R₂ is methyl: 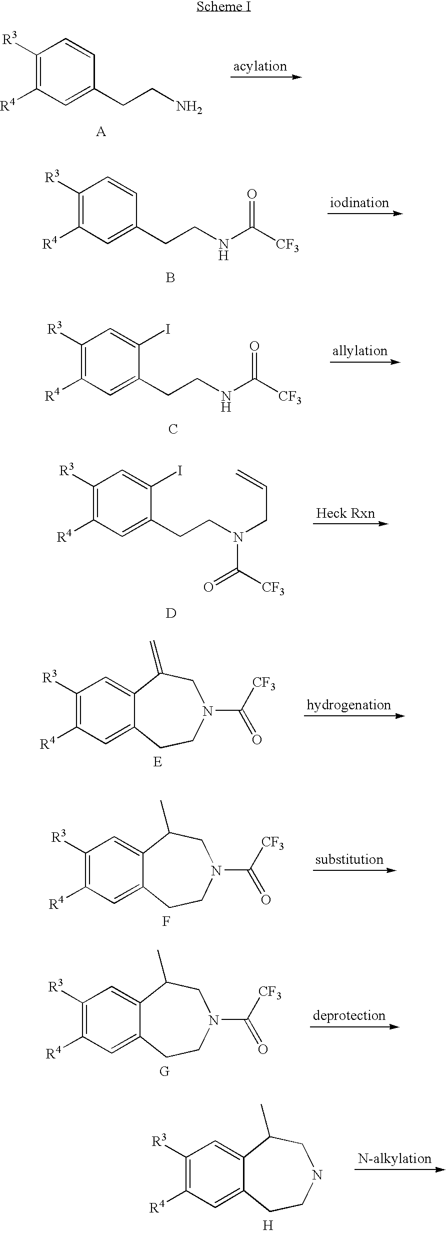
By utilizing, for example, an appropriately substituted 2-phenyl ethylamino Compound A having any of a wide variety of substituents R₃ and R₄, the corresponding substituted 1-methyl-2,3,4,5-tetrahydro-1H-3-benzazepine (Compound H) can be prepared. In a subsequent step, Compound H can be readily alkylated by, for example, treatment with excess paraformaldehyde (for methylation) or a higher order aldehyde, followed by reduction with NaBH₃CN or similar reducing agent according to methodologies known in the art.

Another representative synthetic pathway for the preparation of compounds of the invention is set forth below in Scheme II: 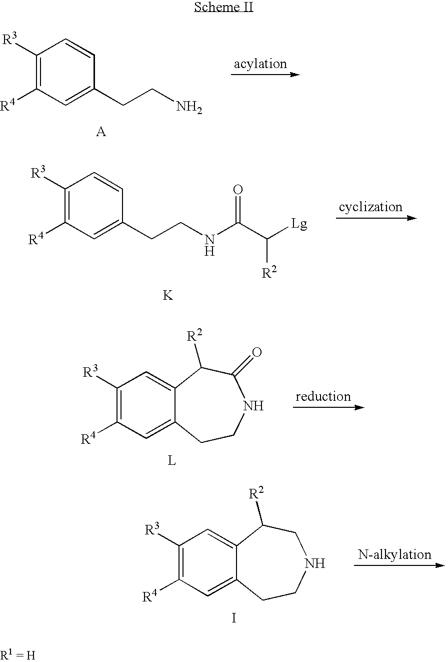
By utilizing, for example, an appropriately substituted 2-phenyl ethylamino Compound A having any of a wide variety of substituents R₃ and R, the corresponding 1-substituted-2,3,4,5-tetrahydro-1H-3-benzazepine [i.e., a compound of Formula (I)] can be prepared. Scheme II illustrates one general pathway for the introduction of R₂ groups into the compounds of the present invention. Compound A is acylated with a carboxylic acid derivative using one of the many methods, such as one of the commonly known coupling agents, available to the artisan. A particularly useful method uses an acid chloride. The carboxylic acid derivative is selected to possess a leaving group or a moiety that can be converted into a leaving group (i.e., Lg). The resulting Compound K is cyclized in the presence of a Lewis Acid, such as, for example, aluminum chloride. After reduction, compounds of the invention are obtained wherein R₁ is H.

In a similar manner as described above in Scheme II compounds of the invention can be prepared by the method shown in Scheme IIa utilizing Compound M. In this method, the alcohol of Compound M is converted into a leaving group (LG) using any number of the methods known in the art to give Compound M′. Suitable reagents include, but not limited to, phosphoryl bromide, phosphoryl chloride, triflic anhydride, triflic chloride, and the like. Compound M′ can be generated in situ or isolated. The R₂ group is subsequently introduced via N-alkylation of an appropriately substituted amino alcohol to give Compound M″. The alcohol of Compound M″ is converted to a suitable leaving group for a Lewis Acid mediated cyclization reaction. Suitable leaving groups include halogens, such as, Br, Cl and the like, but other groups may also be used. The resulting Compound M′″ can be cyclized in the presence of a Lewis Acid, such as, aluminum chloride and the like.

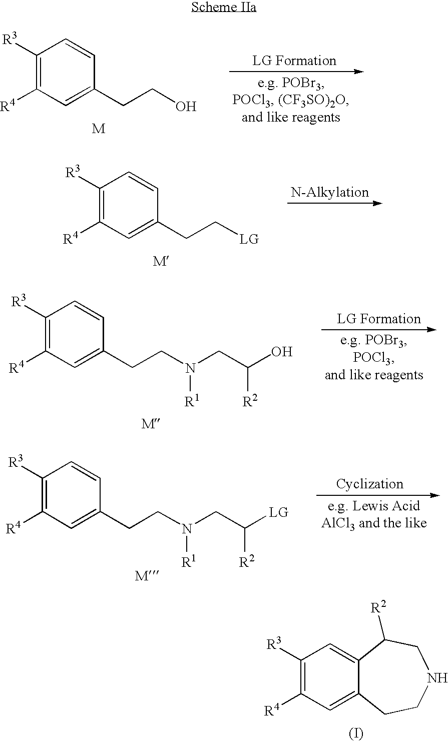

An alternate synthetic approach that can be used to prepare compounds of the present invention utilizes Compound L (i.e., R₂ is H). In this method, the amide nitrogen is first alkylated (providing the R₁ group, Compound N) or protected (i.e., Compound O) using any number of the methods known in the art. The R₂ group is subsequently introduced via an alkylation reaction to provide Compounds P and Q respectively. Alkylation reactions can be conducted under basic conditions, for example, using DMF/NaH, and an alkylating agent of the formula R₂-Lg (wherein: R₂ has the same meaning as described herein and Lg is a leaving group known in the art, such as, Cl, Br, I, OMs, OTs and the like). Examples of the alkylating agent include, but are not limited to, CH₃I, CH₃OMs, CH₃OTs, CH₃CH₂I, CF₃CH₂I, CF₃I, CH₃OCH₂Cl and the like. A representative alkylation example has been reported by Orito, K. and Matsuzaki, T. in Tetrahedron, 1980, 36, 81, 1017-1021, incorporated herein by reference in its entirety. In the example when the nitrogen is protected (i.e., Compound Q), the protecting group is first removed and the amide reduced to provide compounds of the invention wherein R₁ is H. In the example where the nitrogen is alkylated (i.e., Compound P, then the amide is merely reduced to provide compounds wherein R₁ is alkyl. This method is illustrated in Schemes III and IV below.

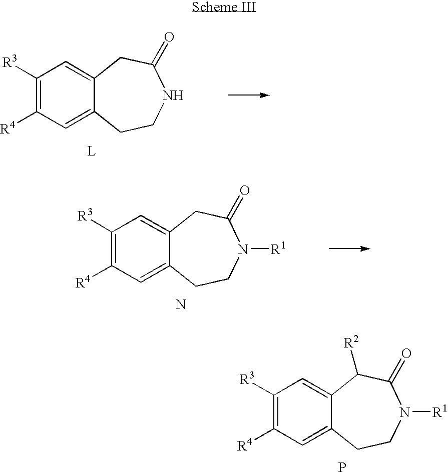

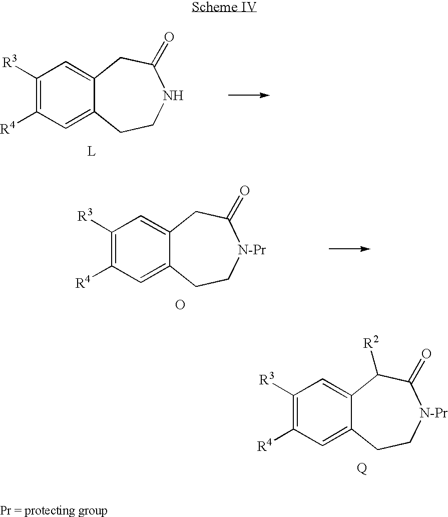

Those of skill in the art will appreciate that a wide variety of compounds of the present invention can be prepared according to Schemes I through IV.

Methods

Compounds of the invention can modulate activity of the 5HT_{2C} receptor. The term “modulate” is meant to refer to an ability to increase or decrease activity of a receptor. Accordingly, compounds of the invention can be used in methods of modulating a 5HT_{2C} receptor by contacting the receptor with any one or more of the compounds described herein. In some embodiments, compounds of the present invention increase activity of the 5HT_{2C} receptor. In further embodiments, compounds of the invention are agonists of the 5HT_{2C} receptor. “Agonists,” as used herein, refer to agents that can stimulate activity (i.e., activate) of a target receptor (e.g., 5HT_{2C}). In further embodiments, the compounds of the invention can be used to modulate a target receptor in an individual in need of modulation of said receptor by administering a therapeutically effective amount of a compound of Formula (I).

The term “contacting” refers to the bringing together of indicated moieties in an in vitro system or an in vivo system. For example, “contacting” a 5HT_{2C} receptor with a compound of the invention includes the administration of a compound of the present invention to an individual or patient, such as a human, having a 5HT_{2C} receptor, as well as, for example, introducing a compound of the invention into a sample containing a cellular or purified preparation containing a 5HT_{2C} receptor. As used herein, the term “individual” or “patient,” used interchangeably, refers to any animal, including mamrnals, preferably mice, rats, other rodents, rabbits, dogs, cats, swine, cattle, sheep, horses, or primates, and most preferably humans.

Another aspect of the present invention pertains to methods of treatment (including prophylaxis) of a 5HT_{2C} receptor-associated disease in an individual (e.g., patient) comprising administering to the individual in need of such treatment a therapeutically effective amount or dose of a compound of the present invention or a pharmaceutical composition thereof.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or syxhptomatology of the disease, condition or disorder (i.e., arresting further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

In some embodiments, the 5HT_{2C} receptor associated disease is selected from the group consisting of disorders of the central nervous system; damage to the central nervous system; cardiovascular disorders; gastrointestinal disorders; diabetes insipidus, sleep apnea, or HDL-related condition. In some embodiments, the individual is a mammral. Preferably, the mammal is a human.

In some embodiments, the disorders of the central nervous system are depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension. In further embodiments, the disorder of the central nervous system is obesity. In further embodiments, the disorder of the central nervous system is Alzheimer's disease. In further embodiments, the sexual dysfunction is male erectile dysfunction.

In some embodiments, the damage to the central nervous system is by trauma, stroke, neurodegenerative diseases, toxic CNS diseases or infective CNS diseases. In further embodiments, the damage to the central nervous system is by encephalitis or meningitis.

In some embodiments, the cardiovascular disorder is thrombosis.

In some embodiments, the gastrointestinal disorder is dysfunction of gastrointestinal motility.

In some embodiments, the HDL-related condition is hypo-HDL related atherosclerotic risk, atherosclerosis, coronary heart disease, ischemic cerebrovascular disease, peripheral vascular disease, stroke or myocardial infarction.

In some embodiments, the 5HT_{2C} receptor-associated related disease is depression, atypical depression, bipolar disorders, anxiety, anxiety disorders, obsessive-compulsive disorders, social phobias, panic states, attention deficit hyperactivity disorder, disruptive behavior disorders, impulse control disorders, borderline personality disorder, sleep disorders (e.g., sleep apnea), autism, seizure disorders, mutism, selective mutism, childhood anxiety disorders, sexual dysfunction in males (e.g., premature ejaculation and erectile difficulty or dysfunction), sexual dysfunction in females, psychoses, schizophrenia, migraine and other conditions associated with cephalic pain or other pain, raised intracranial pressure, epilepsy, personality disorders, Alzheimer's disease, age-related behavioral disorders, behavioral disorders associated with dementia, dementia of aging, organic mental disorders, mental disorders in childhood, aggressivity, age-related memory disorders, memory loss, chronic fatigue syndrome, drug and alcohol addiction, alcoholism, tobacco abuse, weight loss, obesity, bulimia, bulimia nervosa, anorexia nervosa, binge eating disorder, premenstrual tension, premenstrual syndrome (PMS or late luteal phase dysphoric disorder), post-traumatic syndrome, spinal cord injury, damage of the central nervous 'system (e.g., trauma, stroke, neurodegenerative diseases or toxic or infective disorders (e.g., thrombosis), gastrointestinal disorders (e.g., dysfunction of gastrointestinal motility), diabetes insipidus, and type II diabetes.

In some embodiments, the 5HT_{2C} receptor associated disease is selected from the group consisting of high blood pressure, hypertension, high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence), uric acid nephrolithiasis, psychological disorders (such as depression, eating disorders, distorted body image, and low self esteem).

In some embodiments, the 5HT_{2C} receptor-associated disease is selected from the group consisting of psychiatric symptoms and behaviors in individuals with eating disorders such as, but not limited to, anorexia nervosa and bulimia nervosa. Individuals with eating disorders often demonstrate social isolation. For example, anorexic individuals often present symptoms of being depressed, anxious, obsession, perfectionistic traits, and rigid cognitive styles as well as sexual disinterest. In addition to anorexia nervosa and bulimia nervosa, other eating disorders include, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. In essence, what the individual is doing with regard to food and weight is neither normal nor healthy.

In some embodiments, the 5HT_{2C} receptor-associated disease is anorexia athletica (compulsive exercising), body dysmorphic disorder (bigorexia), infection-triggered auto immune subtype of anorexia in children, orthorexia nervosa, night-eating syndrome, nocturnal sleep-related eating disorder, rumination syndrome, gourmand syndrome, Prader-Willi syndrome, pica, or cyclic vomiting syndrome.

Another aspect of the present invention pertains to methods of decreasing food intake of an individual by administering to the individual a therapeutically effective amount or dose of a compound of the present invention or a pharmaceutical composition thereof. In some embodiments, the individual is a mammal. Preferably, the mammal is a human. In fuirther embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of inducing satiety in an individual by administering to the individual a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of controlling weight gain of an individual by administering to the individual suffering from weight control a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In furter embodiments, the human has a body mass index of about 30 to about 45. In further embodiments, the human has a body mass index of about 35 to about 45.

Pharmaceutical Formulations and Dosage Fonns

When employed as pharmaceuticals, the compounds of Formula (I) can be administered in the form of pharmaceutical compositions. These compositions can be administered by a variety of routes including oral, rectal, transdermal, subcutaneous, intravenous, intramuscular, and intranasal, and can be prepared in a manner well known in the pharmaceutical art.

This invention also includes pharmaceutical compositions which contain, as the active ingredient, one or more of the compounds of Formula (I) above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be deterinied by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. An amount adequate to accomplish this is referred to as “therapeutically effective amount.” Effective doses will depend on the disease condition being treated as well as by the judgement of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Some embodiments of the present invention include a method of producing a pharmaceutical composition for “combination-therapy” comprising admixing at least one compound according to any of the compound embodiments disclosed herein, at least one additional pharmaceutical agent, and a pharmaceutically acceptable carrier.

In some embodiments the additional pharmaceutical agent is selected from apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agensts, β₃ adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5-(4-chlorophenyl)-1-(2,4-dichlorophenyl)4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) or appetite suppressants (for example, bupropion). In further embodiments, the additional pharmaceutical agent is orlistat, sibutramine, bromocriptine, ephedrine, leptin, or pseudoephedrine.

In some embodiments the additional pharmaceutical agent is selected from sulfonylureas, meglitinides, biguanides, α-glucosidase inhibitors, peroxisome proliferators-activated receptor-γ(i.e., PPAR-γ) agonists, insulin, insulin analogues, HMG-CoA reducitase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin E receptor antagonists and adiponectin.

It is noted that when the compounds of the invention are utilized as active ingredients in a pharmaceutical composition, these are not intended for use only in humans, but in other non-human mammals as well. Indeed, recent advances in the area of animal health-care mandate that consideration be given for the use of 5HT_{2C} receptor agonists for the treatment of obesity in domestic animals (e.g., cats and dogs), and 5HT_{2C} receptor agonists in other domestic animals where no disease or disorder is evident (e.g., food-oriented animals such as cows, chickens, fish, etc.). Those of ordinary skill in the art are readily credited with understanding the utility of such compounds in such settings.

Combination Therapy

While the compounds of the invention can be administered as the sole active pharmaceutical agent (i.e., mono-therapy), they can also be used in combination with other pharmaceutical agents (i.e., combination-therapy) for the treatment of numerous diseases/conditions/disorders. Therefore, another aspect of the present invention includes methods of treatment comprising administering to an individual in need of prophylaxis and/or treatment a therapeutically effective amount of a compound of the present invention in combination with one or more additional pharmaceutical agents.

As used herein, the phrase “in combination with” is meant to refer to the administration of at least two pharmaceutically active compounds. Typically, the at least two pharmaceutically active compounds include a compound of the invention and an additional pharmaceutical agent. The two pharmaceutically active compounds can be administered together, at the same time, or can be administered sequentially such that both pharmaceutically active compounds have overlapping pharmaceutical effect on the body of the individual receiving the treatment.

Suitable pharmaceutical agents that can be used in combination with the compounds of the present invention include anti-obesity agents such as apolipoprotein-B secretion/microsomal triglyceride transfer protein (apo-B/MTP) inhibitors, MCR4 agonists, cholescystokinin-A (CCK-A) agonists, serotonin and norepinephrine reuptake inhibitors (for example, sibutramine), sympathomimetic agensts, β₃ adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5(4-chlorophenyl)-1-(2,4-dichlorophenyl)4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Other anti-obesity agents, including the agents set forth infra, are well known, or will be readily apparent in light of the instant disclosure, to one of ordinary skill in the art.

In some embodiments, the anti-obesity agents are selected from the group consisting of orlistat, sibutramine, bromocriptine, ephedrine, leptin, and pseudoephedrine. In a further embodiment, compounds of the present invention and combination therapies are administered in combination with exercise and/or a sensible diet.

It will be understood that the scope of combination-therapy of the compounds of the present invention with other anti-obesity agents, anorectic agents, appetite suppressant and related agents is not limited to those listed above, but includes in principle any combination with any pharmaceutical agent or pharmaceutical composition useful for the treatment of overweight and obese individuals.

Other suitable pharmaceutical agents, in addition to anti-obesity agents, that can be used in combination with the compounds of the present invention include agents usefil in the treatment of concomitant diseases. For example, individuals that are over weight or obese increase their risk of morbidity and mortality arising from concomitant diseases, such as, but not limited to, congestive heart failure, type II diabetes, atherosclerosis, dyslipidemia, hyperinsulinemia, hypertension, insulin resistance, hyperglycemia, retinopathy, nephropathy and neuropathy. Treatment for one or more of the diseases cited herein include the use of one or more pharmaceutical agents known in the art belonging to the classes of drugs referred to, but not limited to, the following: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol4owering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In accordance to one aspect of the present invention, a compound of the present can be used in combination with a pharmaceutical agent or agents belonging to one or more of the classes of drugs cited herein.

It will be understood that the scope of combination-therapy of the compounds of the present invention with other pharmaceutical agents is not limited to those listed herein, supra or infra, but includes in principle any combination with any pharmaceutical agent or pharmaceutical composition useful for the treatment diseases, conditions or disorders that are linked to overweight and obese individuals.

Some embodiments of the present invention include methods of treatment of a disease, disorder or condition by administering to an individual in need of such treatment a therapeutically effect amount or dose of a compound of the present invention in combination with at least one pharmaceutical agent selected from the group consisting of: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In some embodiments, methods of the present invention include compounds of the present invention and the pharmaceutical agents are administered separately. In further embodiments, compounds of the present invention and the pharmaceutical agents are administered together.

Suitable pharmaceutical agents that can be used in combination with compounds of the present invention include α-glucosidase inhibitors. α-Glucosidase inhibitors belong to the class of drugs which competitively inhibit digestive enzymes such as α-amylase, maltase, α-dextrinase, sucrase, etc. in the pancreas and or small intesting. The reversible inhibition by α-glucosidase inhibitors retard, diminish or otherwise reduce blood glucose levels by delaying the digestion of starch and sugars. Some representative examples of a-glucosidase inhibitors include acarbose, N-1,3dihydroxy-2-propyl)valiolamine (generic name; voglibose), miglitol, and α-glucosidase inhibitors known in the art.

Suitable pharmaceutical agents that can be used in combination with compounds of the present invention include sulfonylureas. The sulfonylureas (SU) are drugs which promote secretion of insulin from pancreatic β cells by transmitting signals of insulin secretion via SU receptors in the cell membranes. Examples of the sulfonylureas include glyburide, glipizide, glimepiride and other sulfonylureas known in the art.

Suitable pharmaceutical agents that can be used in combination with compounds of the present invention include the meglitinides. The meglitinides are benzoic acid derivatives represent a novel class of insulin secretagogues. These agents target postprandial hyperglycemia and show comparable efficacy to sulfonylureas in reducing HbA_{1c}. Examples of meglitinides include repaglinide, nateglinide and other meglitinides known in the art.

Suitable pharmaceutical agents that can be used in combination with compounds of the present invention include the biguanides. The biguanides represent a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Suitable pharmaceutical agents that can be used in combination with compounds of the present invention include the α-glucosidase inhibitors. The α-glucosidase inhibitors competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Examples of α-glucosidase inhibitors include acarbose, N-(1,3-dihydroxy-2-propyl)valiolarnine (generic name; voglibose), miglitol, and α-glucosidase inhibitors known in the art.

Suitable pharmaceutical agents that can be used in combination with compounds of the present invention include the peroxisome proliferators-activated receptor-γ(i.e., PPAR-γ) agonists. The peroxisome proliferators-activated receptor-y agonists represent a class of compounds that activates the nuclear receptor PPAR-γ and therefore regulate the transcription of insulin-responsive genes involved in the control of glucose production, transport and utilization. Agents in the class also facilitate the regulation of fatty acid metabolism. Examples of PPAR-γ agonists include rosiglitazone, pioglitazone, tesaglitazar, netoglitazone, GW409544, GW-501516 and PPAR-γ agonists known in the art.

Suitable pharmaceutical agents that can be used in combination with compounds of the present invention include the HMIG-CoA reductase inhibitors. The HMG-CoA reductase inhibitors are agents also referred to as Statin compounds that belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. The statins lower serum LDL concentrations by upregulating the activity of LDL receptors and are responsible for clearing LDL from the blood. Some representative examples the statin compounds include rosuvastatin, pravastatin and its sodium salt, simvastatin, lovastatin, atorvastatin, fluvastatin, cerivastatin, rosuvastatin, pitavastatin, BMS's “superstatin”, and HMG-CoA reductase inhibitors known in the art.

Suitable pharmaceutical agents that can be used in combination with compounds of the present invention include the angiotensin converting enzyme (ACE) inhibitors. The angiotensin converting enzyme inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the-art.

Suitable pharmaceutical agents that can be used in combination with compounds of the present invention include the angiotensin II receptor antagonists. Angiotensin II receptor antagonists target the angiotensin II receptor subtype 1 (i.e., AT1) and demonstrate a beneficial effect on hypertension. Examples of, angiotensin II receptor antagonists include losartan (and the potassium salt form), and angiotensin II receptor antagonists known in the art.

Other treatments for one or more of the diseases cited herein include the use of pharmaceutical agents known in the art belonging to the classes of drugs referred to, but not limited to, the following: amylin agonists (for example, pramlintide), insulin secretagogues (for example, GLP-1 agonists; exendin4; insulinotropin (NN2211); dipeptyl peptidase inhibitors (for example, NVP-DPP-728), acyl CoA cholesterol acetyltransferase inhibitors (for example, Ezetimibe, eflucimibe, and like compounds), cholesterol absorption inhibitors (for example, ezetimibe, pamaqueside and like compounds), cholesterol ester transfer protein inhibitors (for example, CP-529414, JTT-705, CETi-1, and like compounds), microsomal triglyceride transfer protein inhibitors (for example, implitapide, and like compounds), cholesterol modulators (for example, NO-1886, and like compounds), bile acid modulators (for example, GT103-279 and like compounds) and squalene synthase inhibitors.

Squalene synthesis inhibitors belong to a class of drugs that lower blood cholesterol levels by inhibiting synthesis of squalene. Examples of the squalene synthesis inhibitors include (S)-α-[Bis [2,2-dimethyl-1-oxopropoxy)methoxy] phosphinyl]-3-phenoxybenzenebutanesulfonic acid, mono potassium salt (BMS-188494) and squalene synthesis inhibitors known in the art.

Labeled Compounds and Assay-type Methods

Another aspect of the present invention relates to radio-labeled compounds of Formula (I) that would be useful not only in radio-imaging but also in assays, both in vitro and in vivo, for localizing and quantitating the 5HT_{2C} receptor in tissue samples, including human, and for identifying 5HT_{2C} receptor ligands by inhibition binding of a radio-labeled compound. Accordingly, the present invention includes 5HT_{2C} receptor assays that contain such radio-labeled compounds.

The present invention further includes isotopically-labeled compounds of Formula (I). An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶CI, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio4abeled compound. For example, for in vitro 5HT_{2C} receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes supra and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the more scarce radio-isotope or nonradio-active isotope.

Synthetic methods for incorporating radio-isotopes into organic compounds are applicable to compounds of the invention and are well known in the art. For example, methods of incorporating tritium into target molecules, are as follows:

A. Catalytic Reduction with Tritium Gas—This procedure normally yields high specific activity products and requires halogenated or unsaturated precursors;

B. Reduction with Sodium Borohydride [³H]—This procedure is typically inexpensive and requires precursors containing reducible functional groups such as aldehydes, ketones, lactones, esters, and the like;

C. Reduction with Lithium Aluminum Hydride [³H]—This procedure offers products at almost theoretical specific activities and requires precursors containing reducible functional groups such as aldehydes, ketones, lactones, esters, and the like;

D. Tritium Gas Exposure Labeling—This procedure involves exposing precursors containing exchangeable protons to tritium gas in the presence of a suitable catalyst; and

E. N-Methylation using Methyl Iodide [³ H]—This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Example synthetic methods for incorporating activity levels of ¹²⁵I into target molecules include:

A. Sandmeyer and like reactions—This procedure transforms an aryl or heteroaryl amine into a diazonium salt, such as a tetrafluoroborate salt, and subsequently to ¹²⁵I labeled compound using Na¹²⁵I. A representative procedure is reported by Zhu, D.-G. et al., J Org. Chem. 2002, 67, 943-948;

B. Ortho ¹²⁵Iodination of phenols—This procedure allows for the incorporation of ¹²⁵I at the ortho position of a phenol as reported by Collier, T. L. et al., J. Labeled Compd Radiopharm. 1999, 42, S264-S266; and

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported in Bas, M.-D. et al., J. Labeled Compd Radiopharm. 2001, 44, S280-S282.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the 5HT_{2C} receptor. Accordingly, the ability of a test compound to compete with the radio-labeled compound for binding to the 5HT_{2C} receptor directly correlates to its binding affinity.

Labeled compounds of the present invention bind to the 5HT_{2C} receptor. In one embodiment the labeled compound has an IC₅₀ less than about 500 μM, in another embodiment the labeled compound has an ICso less than about 100 μM, in yet another embodiment the labeled compound has an IC₅₀ less than about 10 μM, in yet another embodiment the labeled compound has an IC₅₀ less than about 1 pM, and in still yet another embodiment the labeled inhibitor has an IC₅₀ less than about 0.1 μM.

Other uses of the disclosed receptors and methods will become apparent to those in the art based upon, inter alia, a review of this disclosure.

Kits

The present invention also includes pharmaceutical kits useful, for example, in the treatment or prevention of 5HT_{2C}-related diseases, which include one or more containers containing a pharmaceutical composition comprising a therapeutically effective amount of a compound of Formula (I). Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example A

### N-Trifluoroacetyl-4-chlorophenethylainine

A solution of 4-chlorophenethylamine (1.0 g, 6.4 mmol) in dichloromethane (20 mL) was cooled to 0° C., treated with pyridine (1.0 mL, 12.8 nmmol), trifluoracetic anhydride (1.6 g, 7.7 mmol) and then stirred for 1 hour while warming to 20° C. The product mixture was diluted with EtOAc (100 mL), washed sequentially with 10% aqueous HCl (50 mL), water (50 mL), brine (50 mL), dried with Na₂SO₄ and concentrated to give 1.6 g of a white solid.

### N-Trifluoroacetyl-2-iodo-4-chlorophenethylamiiie

A solution of N-tritluoroacetyl-4-chlorophenethylamine (1.6 g, 6.4 mmol) in dichloromethane (20 mL) was treated with bis(pyridine)iodonium(I)tetrafluoroborate (2.6 g, 7.0 mmol), CF₃SO₃H (2.1 g, 14.1 mmol) and stirred overnight at 20° C. The product mixture was concentrated, dissolved in EtOAc (100 mL), washed twice with 5% aqueous sodium bisulfite (50 mL), twice with saturated aqueous NaHCO₃, (50 mL) once with brine (50 mL), dried with Na₂SO₄ and concentrated to give 0.94 g of a clear oil. MS calculated for C₁₀H₈ClF₃INO+H: 378, observed: 378.

### N-Allyl, N-tnifluoroacetyl-2-iodo-4-chlorophenethylamine

A solution of N-trifluoroacetyl-2-iodo-4-chlorophenethylamine (0.94 g, 2.4 minol) in toluene (25 mL) was treated with K₂CO₃ (0.43 g, 3.12 mmol), KOH (0.40 g, 7.2 mmol), n-Bu₄NBr (0.077 g, 0.24 mmol) and allyl bromide (0.43 g, 3.6 mmol) sequentially. The mixture was stirred at 80° C. for 3.5 hours, cooled to 20° C. and acidified with 10% aqueous HCl. The phases were separated, the aqueous phase extracted with ether (100 mL), the combined organic phases were washed with brine (50 mL), dried with Na₂SO₄ and concentrated to give 0.76 g of a clear oil. MS calculated for C₁₃H₁₂ClF₃INO+H: 418, observed: 418.

### N-Trifluoroacetyl-8-chloro-1-methylene-2,3,4,5-tetrahydro-1H-3-benzazepine

A solution of N-allyl, N-trifluoroacetyl-2-iodo-4-chlorophenethylamine (0.76 g, 1.8 mmol) in dimethylformamide (20 mL) was treated with KOAc (0.53 g, 5.4 mmol), n-Bu₄NBr (0.58 g, 1.8 mmol), PPh₃ (0.047 g, 0.18 mmol), Pd(OAc)₂ (0.041 g, 0.18 mmol) and stirred overnight at 105° C. The product mixture was cooled to 20° C., filtered, diluted with water (100 mL), extracted with ether (3×100 mL), the combined organic phases washed with water (100 mL), brine (100 mL), dried with Na₂SO₄ and concentrated. Flash chromatography (10% EtOAc in hexane, silica) resulted in 0.228 g of a clear oil. ¹H NMR (400 MHz, CDCl₃) d 7.29 (s, 1H), 7.18 (m, 1H), 7.04 (m, 1H), 5.38 (m, 2H), 5.40 (d, J=16 Hz, 2H), 3.80 (m, 2H), 3.00 (m, 2H). MS calculated for C₁₃H₁₁ClF₃NO+H: 290, observed: 290.

### N-Trifluoroacetyl-8-chloro-1-methyl-2,3,4,5-tetrahydro-]1H-3-benzazepine

A solution of N-trifluoroacetyl-8-chloro-1-methylene-2,3,4,5-trihydro-1H-3-benzazepine (0.16 g, 0.55 mmol) in methanol (10 mL) was treated with 10% Pd/C (0.02 g) and stirred 30 minutes under an atmosphere of hydrogen. The product mixture was filtered, concentrated and purified by flash chromatography (5% EtOAc in hexane, silica) resulting in 0.057 g of a white solid. MS calculated for C₁₃H₁₃ClF₃NO+H: 292, observed: 292.

### Chiral Column Resolution

N-Trifluoroacetyl-8chloro-1-methyl-2,3,4,5-tetrahydro-1H-3-benzazepine was separated into its corresponding enantiomers using CHIRALCEL® OD® with 95/5 hexane/isopropanol (detection is UV at 245 nm).

### (S)-8-chloro-1-methyl-2,3,4,5-tetraliydro-1H-3-benzazepine

To a solution of (S)-N-trifluoroacetyl-8-chloro-1-methyl-2,3,4,5-tetrahydro-1H-3-benzazepine (15.4 g, 52.9 mmol) in methanol (200 mL) stirred at room temperature was added 50% aqueous NaOH. (8.48 g, 106 mmole) over about ten minutes. The resulting mixture was stirred at room temperature for 2.5 hours, at which point LC/MS analysis of the reaction mixture revealed complete conversion of starting trifluoroacetamide to the title amine. Solvent was then rotary evaporated from the reaction mixture at reduced pressure with a 25° C. water bath. The solid evaporation residue was partitioned between dichloromethane (150 mL) and water (75 mL). The aqueous phase was separated and extracted with dichloromethane (30 mL). The two dichloromethane extracts were combined, washed with brine (two 80-mL portions), and rotary evaporated at reduced pressure with a 25° C. water bath. The resulting solid residue was almost completely dissolved in hexane (75 mL, room temperature), and the hexane solution was filtered and rotary evaporated at reduced pressure with a 25° C. water bath to a solid residue of the title amine (10.28 g, 99% yield).

### Example 1

### (S)-Benzyl-(5-methyl-2,3,4,5-tetrahydro-1H-benzo[d]azepin-7-yl)-amine hydrochloride

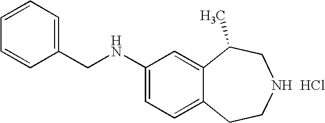

### (S)-N-tertbutoxycarbonyl-8-chloro-1-metlyl-2,3,4,5-tetrahydro-1H-beiizo[d]azepine

A solution of (S)-8chloro-1-methyl-2,3,4,5-tetrahydro-1H-benzo[d]azepine (1.69 g, 7.28 mmol) in methanol (50 mL) was treated with di-tert-butyl dicarbonate (1.59 g, 7.28 mmol), triethylamine (1.47 g, 14.6 mmol) and stirred at 25° C. for 2 hours. The mixture was diluted with 3:1 EtOAc/hexane (100 mL), washed with saturated aqueous citric acid (100 mL), washed with water (50 mL), dried with MgSO₄ and concentrated. MS calculated for C₁₆H₂₂ClNO₂+H: 296, observed: 296.

### (S)-Benzyl-(5-methyl-2,3,4,5-tetrahydro-1H-benzo[d]azepin-7-yl)-anzine hydrochloride

A solution of (S)-N-tertbutoxycarbonyl-8-chloro-1-methyl-2,3,4,5-tetrahydro-1 H-benzo[d]azepine (75 mg, 0.254 mmol) in toluene (2.0 mL) was treated with benzylamine (41 mg, 0.38 mmol), NaOtBu (34 mg, 0.35 mmol), Pd(OAc)₂ (3 mg, 0.013 mmol), 2-(di-t-butylphosphino)biphenyl (8 mg, 0.025 mmol) and heated to 140° C. for 20 minutes by microwave. The product mixture was filtered through celite and silica and then concentrated. Flash chromatography (10% EtOAc in hexane, silica) resulted in 78 mg of a clear oil. MS calculated for C₂₃H₃₀N₂O₂+H: 367, observed: 367. The product (78 mg, 0.224 mmol) was dissolved in 2:1 dichloromethane/methanol (1.5 mL) treated with 2.0 M HCl in ether (0.5 mL), and stirred overnight at 20° C. The product mixture was evaporated to give 64 mg of the HCl salt as a white solid. ¹H NMR (400 MHz, CD₃OD) δ 7.44 (m, 5H), 7.42 (d, J=8 Hz, 1H), 7.31 (d, J=8Hz, 1H), 7.23 (s, 1H), 4.61 (s, 2H), 3.50 (m, 2H), 3.37 (m, 2H), 3.18 (dd, J=9, 16 Hz, 1H), 3.08 (dd, J=12, 12 Hz, 1H), 2.97 (dd, J=9, 12 Hz, 1H), 1.40 (d, J=7 Hz, 3H). MS calculated for C₁₈H₂₂N₂+H: 267, observed: 267.

### Example 2

### (S)-Benzyl-methyl-(5-methyl-2,3,4,5-tetrahydro-1H-benzo[d]azepin-7-yl)-amine hydrochloride

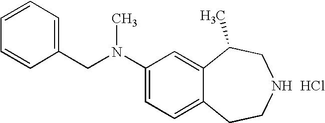

By the same general procedure as Example 1, (S)-benzyl-methyl-(5-methyl-2,3,4,5-tetrahydro-1H-benzo[d]azepin-7-yl)-amine hydrochloride was obtained from N-methylbenzylamine as a crystalline solid. ¹H NMR (400 MHz, CD₃OD) δ 7.51 (d, J=7 Hz, 1H), 7.40-7.29 (m, 7H), 4.81 (s, 2H), 3.50-3.46 (m, 2H), 3.40 (s, 3H), 3.37-3.30 (m, 2H), 3.17 (dd, J=6, 16 Hz, 1H), 3.04 (dd, J=12, 12 Hz, 1H), 2.92 (m, 1H), 1.37 (d, J=7 Hz, 3H). MS calculated for C₁₉H₂₄N₂+H: 281, observed: 281.

### Example 3

### (5-S,1′-R)-(5-Methyl-2,3,4,5-tetrahydro-1H-benzo[d]azepin-7-yl)-(1′-pheiyl-ethyl)-amine hydrochloride

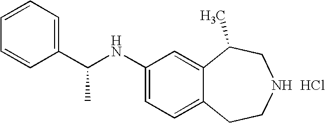

By the same general procedure as Example 1, 5-S,1′-R-5-methyl-2,3,4,5-etrahydro-1H-benzo[d]azepin-7-yl)-(1-phenyl-ethyl)-amine hydrochloride was obtained from (R)-1-phenylethylamine as a crystalline solid. ¹H NMR (400 MHz, CD₃OD) δ 7.41 (m, 5H), 7.36 (d, J=8 Hz, 1H), 7.24 (d, J=8 Hz, 1H), 7.06 (s, 1H), 4.80 (m, 1H), 3.48-3.40 (m, 2H), 3.36-3.27 (m, 2H), 3.14 (dd, J=6, 16 Hz, 1H), 3.03 (dd, J=12, 12 Hz, 1H), 2.92 (m, 1H), 1.82 (d, J=7 Hz, 3H), 1.33 (d, J=7 Hz, 3H). MS calculated for C₁₉H₂₄N₂+H: 281, observed: 281.

### Example 4

### (5-S,1′-S)-(5-Methyl-2,3,4,5-tetrahydro-1H-benzo[d]azepin-7-yl)-(1′-phenyl-ethyl)-amine hydrochloride

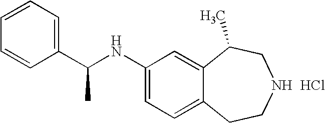

By the same general procedure as Example 1, 5-S,1′S-(5-methyl-2,3,4,5-tetrahydro-1H-benzo[d]azepin-7-yl)-(1-phenyl-ethyl)-amine hydrochloride was obtained from (S)-1-phenylethylamine as a crystalline solid. ¹H NMR (400 MHz, CD₃OD) δ 7.39 (m, 5H), 7.33 (d, J=8 Hz, 1H), 7.25 (d, J=8 Hz, 1H), 7.07 (s, 1H), 4.80 (m, 1H), 3.49-3.45 (m, 2H), 3.36-3.29 (m, 2H), 3.13 (dd, J=6, 16 Hz, 1H), 3.01 (dd, J=12, 13 Hz, 1H), 2.83 (dd, J=9, 13 Hz, 1H), 1.85 (d, J=7 Hz, 3H), 1.33 (d, J=7 Hz, 3H). MS calculated for C₁₉H₂₄N₂+H: 281, observed: 281.

### Example 5

### (S)-(5-Methyl-2,3,4,5-tetrahydro-1H-benzo[d]azepin-7-yl)-phenyl-amine hydrochloride

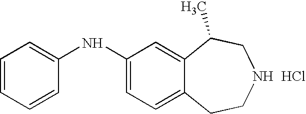

By the same general procedure as Example 1, (S)-5-methyl-2,3,4,5-tetrahydro-1H-benzo[d]azepin-7-yl)-phenyl-amine hydrochloride was obtained from aniline as a crystalline solid. ¹H NMR (400 Mz, CD₃OD) δ 7.55-7.50 (m, 2H), 7.41 (d, J=8 Hz, 1H), 7.29 (dd, J=8, 8 Hz, 2H), 7.15 (dd, J=8, 8 Hz, 2H), 7.09 (s, 1H), 7.03-6.99 (m, 2H), 3.41-3.34 (m, 2H), 3.18 (m, 1H), 3.12-3.06 (m, 3H), 1.44 (d, J=7 Hz, 3H). MS calculated for C₁₇H₂₀N₂+H: 253, observed: 253.

### Example 6

### (R,S)-(5-Methyl-2,3,4,5-tetrahydro-1H-benzo[d]azepin-7-yl)phenethyl-amine hydrochloride

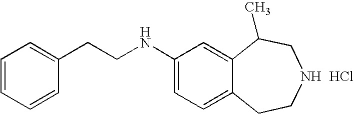

By the same general procedure as Example 1, (5-methyl-2,3,4,5-tetrahydro-1H-benzo[d]azepin-7-yl)-phenethyl-amie hydrochloride was obtained from phenethylamine as a crystalline solid. ¹H NMR (400 MHz, CD₃OD) δ 7.50 (s, 1H), 7.45 (s, 2H), 7.32-7.25 (m, 5H), 3.68-3.64 (m, 2H), 3.58-3.45 (m, 2H), 3.42-3.32 (m, 2H), 3.18 (dd, J=7, 16 Hz, 1H), 3.14-3.00 (m, 4H), 1.51 (d, J=7 Hz, 3H). MS calculated for C₁₉H₂₄N₂+H: 281, observed: 281.

### Example 7

### (S)-(5-Methyl-2,3,4,5-tetrahydro-1H-benzo[d]azepin-7-yl)-(3-phenyl-propyl)-amine hydrochloride

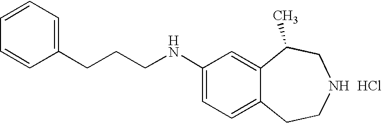

By the same general procedure as example 1, (S)-(5-methyl-2,3,4,5-tetrahydro-1H-benzo[d]azepin-7-yl){3-phenyl-propyl)-amine hydrochloride was obtained from 3-phenylpropylamine as a crystalline solid. ¹H NMR (400 MHz, CD₃OD) δ 7.45-7.42 (m, 2H), 7.35 (d, J=8 Hz, 1H), 7.30-7.27 (m, 2H), 7.22-7.19 (m, 3H), 3.41-3.31 (m, 6H), 3.21 (dd, J=7, 16 Hz, 1H), 3.17-3.00 (m, 4H), 2.75 (dd, J=8, 8 Hz, 2H), 2.07 (M, 2H), 1.50 (d, J=7 Hz, 3H). MS calculated for C₂₀H₂₆N₂+H: 295, observed: 295.

### Example 8

### (R,S)-7-Benzyloxy-1-methyl-2,3,4,5tetrahydro-1H-benzo [d]azepine hydrochloride

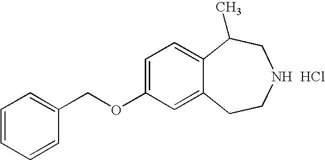

### 2-Chloro-N-[2-(4-methoxyphenylphenyl)ethyl]propionamide

A solution of 4-methoxyphenethylamine (0.80 g, 5.3 mmol) in dichloromethane (20 mL) was treated with diisopropylethylamine (0.82 g, 6.3 mmol) and 2-chloropropionylchloride (0.67 mL, 5.3 mmol) sequentially, and stirred at 20 ° C. for 4 hours. The niixture was diluted with dichloromethane (50 mL), washed with 10% aqueous HCl, brine (20 mL), dried with Na₂SO₄ and concentrated, resulting in 1.5 g of a brown oil. ¹H NMR (400 MHz, CDCl₃) δ 7.09 (d, J=10 Hz, 2H), 6.85 (d, J=10 Hz, 2H), 6.57 (bs, 1H), 4.36 (q, J=7 Hz, 1H), 3.79 (s, 3H), 3.49 (dd, J=7, 7 Hz, 2H), 2.77 (dd, J=7, 7 Hz, 2H), 1.69 (d, J=7 Hz, 3H).

### 7-hydroxy-1-methyl-2-oxo-2,3,5-trihydro-1H-benzo[d]azepine

Neat 2-chloro-N-[2-(4-methoxyphenyl)ethyl]propionamide (2.2 g, 9.1 mmol) and AlCl₃ (3.6 g, 27 mmol) were heated at 150° C. for 18 hours while stirring. The product mixture was quenched with water (10 mL), diluted with dichloromethane (100 mL), the organic phase separated, washed with brine (50 mL), dried with Na₂SO₄ and concentrated, resulting in 1.7 g of a brown oil. ¹H NMR (400 MHz, CD₃OD) δ 7.03 (d, J=8 Hz, 1H), 6.63 (d, J=8 Hz, 1H), 6.60 (s, 1H), 4.20 (q, J=7 Hz, 1H), 3.81-3.74 (m, 1H), 3.39-3.32 (m, 1H), 3.25-3.18 (m, 1H), 3.02-2.94 (m, 1H), 1.47 (d, J=7 Hz, 3H).

### 7-benzyloxy-1-methyl-2-oxo-2,3,5-trihydro-1H-benzo[d]azepine

A solution of 7-hydroxy-1-methyl-2-oxo-2,3,5-trihydro-1H-benzo[d]azepine (0.041 g, 0.213 mmol) in dichloromethane (5 mL) was treated with benzyl bromide (0.072 g, 0.64 mmol), DBU (0.100 g, 0.64 mmol), and stirred 2 hours at 20° C. The product mixture was diluted with EtOAc (50 mL), washed with 5% aqueous HCl (20 mL), brine (20 mL), dried with Na₂SO₄ and concentrated. Flash chromatography (15% EtOAc in hexane, silica) resulted in 0.045 g of a clear oil. ¹H NMR (400 MHz, CDCl₃) δ 7.41-7.28 (m, 5H), 7.13 (d, J=8 Hz, 1H), 6.80 (d, J=8 Hz, 1H), 6.75 (s, 1H), 5.98 (bs, 1H), 5.02 (s, 2H), 4.12 (q, J=7 Hz, 1H), 3.79-3.71 (m, 1H), 3.41-3.33 (m, 1H), 3.30-3.24 (m, 1H), 3.22-2.95 (m, 1H), 1.53 (d, J=7 Hz, 3H).

### (R,S)-7-benzyloxy-1-methyl-2,3,4,5-tetrahydro-1H-benzo[d]azepine hydrochloride

A solution of 7-benzyloxy-1-methyl-2-oxo-2,3,5-trihydro-1H-benzo[d]azepine (45 mg, 0.160 mmol) in tetrahydrofuran (1 mL) was treated with 1.0 M borane in THF (0.48 mL, 0.480 mmol), and stirred at 20° C. for 5 hours. The mixture was quenched with methanol (0.1 mL), acidified with concentrated HCl (0.1 mL), azeotroped with methanol (3×2 mL) and concentrated. Flash chromatography (5% methanol in dichloromethane) resulted in 31 mg of a clear oil. The compound was treated with 1.0 N HCl in ether (1 mL) evaporated to give 31 mg of white solid. ¹H NMR (400 MHz, CD₃OD) δ 7.41-7.25 (m, 5H), 7.16 (d, J=8 Hz, 1H), 6.87 (d, J=8 Hz, 1H), 6.86 (s, 1H), 5.06 (s, 2H), 3.40 (m, 1H), 3.33 (m, 2H), 3.18 (m, 1H), 3.06 (m, 2H), 2.97 (m, 1H), 1.42 (d, J=7 Hz, 3H). MS calculated for C₁₈H₂₁NO+H: 268, observed: 268.

### Example 9

### (S)-1-Methyl-8-phenyl-2,3,4,5-tetrahydro-1H-benzo[d]azepine hydrochloride

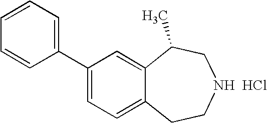

A solution of (S)-N-t-butoxycarbonyl-8-chloro-1-methyl-2,3,4,5-tetrahydro-1H-benzo[d]azepine. (95 mg, 0.32 mmol) in 1,4-dioxane (1.0 mL) was treated with phenyltributyl stannane (123 mg, 0.33 mmol), CsF (107 mg, 0.70 mmol), Pd(PtBu₃)₂ (8 mg, 0.02 mmol) and then stirred 16 hours at 100° C. The product mixture was cooled, diluted with EtOAc, filtered through celite, concentrated and purified by flash chromatography (10% EtOAc in hexane, silica) which resulted in 36 mg of clear oil. The intermediate (36 mg; 0.11 mmol) was dissolved in methanol (2 mL) treated with 4.0 HCl in dioxane (1 mL), and stirred one hour at 20° C. The compound was treated with 1.0 N HCl in ether (1 mL) evaporated to give 25 mg of white solid. ¹H NMR (400 MHz, DMSO-d₆) δ 7.60 (d, J=8 Hz, 2H), 7.42 (m, 4H), 7.33 (dd, J=8, 8 Hz, 1H) 7.25 (d, J=8 Hz, 1H), 3.45 (m, 1H), 3.36 (m, 1H), 3.28-3.16 (m, 3H), 3.03 (dd, J=7, 16 Hz, 1H), 2.95 (m, 2H), 1.39 (d, J=8 Hz, 3H). MS calculated for C₁₇H₁₇N+H: 238, observed: 238.

### Example 10

### (R,S)-1-Methyl-7-(1-phenyl-ethoxy)-2,3,4,5-tetrahydro-1H-benzo[d]azepine hydrochloride

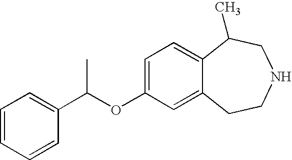

By the same general procedure as Example 8, 1-methyl-8-(1-phenyl-ethoxy)-2,3,4,5-tetrahydro-1H-benzo[d]azepine hydrochloride was obtained from (1-bromoethyl)benzene as a crystalline solid. MS calculated for C₁₉H₂₃NO+H: 282, observed: 282.

### Example 11

### (R,S)-1-Methyl-7-phenethyloxy-2,3,4,5-tetrahydro-1H-benzo[d]azepine hydrochloride

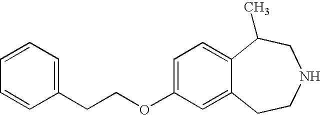

By the same general procedure as Example 8, 1-methyl-8-phenethyloxy-2,3,4,5-tetrahydro-1H-benzo[d]azepine hydrochloride was obtained from 2-phenylethyl bromide as a crystalline solid. MS calculated for C₁₉H₂₃NO+H: 282, observed: 282.

### Example 12

### (R,S)-1-Methyl-7-(3-phenyl-propoxy)-2,3,4,5-tetrahydro-1H-benzo[d] azepine hydrochloride

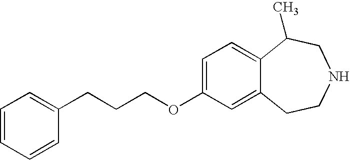

By the same general procedure as Example 8, 1-methyl-8-(3-phenyl-propoxy)-2,3,4,5-tetrahydro-1H-benzo[d]azepine hydrochloride was obtained from 3-phenylpropyl bromide as a crystalline solid. MS calculated for C₂₀H₂₅NO+H: 296, observed: 296.

### Example 13

### (R,S-8-Blenzyloxy-1-methyl-2,3,4,5-tetrahydro-1H-benzo[d]azepine

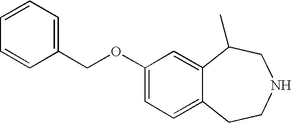

### 1-[2-(4-Methoxy-phenyl)-ethylamino]-propan-2-ol

To a solution of 4-methoxyphenethyl alcohol (6.6 mmol, 1.0 g) in dichloromethane (50 mL) was added diisopropylethyl amine (19.7 mmol, 3.43 mL) and mesylchloride (13.2 mmol, 1.02 mL). This was stirred for 1 h, diluted with dichloromethane (100 mL) and partitioned with water (75 mL). The organics were washed with saturated NaHCO₃ (50 mL) and brine (50 mL), dried with Na₂SO₄, filtered and concentrated. The crude material was treated with neat 1-amino-2-propanol (5 mL) and heated for 3 h. This was diluted with water (50 mL) and extracted with EtOAc (2×75 mL). The combined organics were washed with brine (50 mL), dried with Na₂SO₄, filtered and concentrated. The crude amine was dissolved in dichloromethane (20 ml) and 2.0 M HCl in ether (5 ml) added to form a white precipitate which was collected by filtration. The white solid was partitioned between EtOAc (100 ml) and saturated NaHCO₃ (50 mL). The organics were dried with Na₂SO₄, filtered and concentrated to furnish 900 mg of 1-[2-4-Methoxy-phenyl)-ethylamino]-propan-2-ol. ¹H NMR (HCl salt) (400 MHz, DMSO) δ 9.20 (br.s, 1H), 8.87 (br.s, 1H), 7.17 (d, J=9 Hz, 1H), 6.89 (d, J=9 Hz, 1H), 4.03-3.99 (m, 1H), 3.73 (s, 3H), 3.10-2.91 (m, 5H), 2.80-2.76 (m, 1H), 1.12 (d, J=6 Hz, 3H).

### (2-Bromo-propyl)-[2-(4-methoxy-phenyl)-ethyl]-amine hydrobromide

To a solution of 1-[2-(4-methoxy-phenyl)-ethylamino]-propan-2-ol (4.3 mmol, 0.90 g) in dichloromethane (20 mL) at 0° C. was added dimethyl formamide (2.2 mmol, 0.241 g) and thionyl bromide (5.6 mmol, 0.67 ml). This was stirred for 14 h while warming to 20° C. Diethyl ether (10 mL, 0° C.) was added and the reaction cooled to 0° C. The precipitate that was formed was collected by filtration to give 0.688 g of an off-white solid. ¹H NMR (400 MHz, DMSO) δ 8.89 (br.s, 1H), 8.78 (br.s, 1H), 7.18 (d, J=9 Hz, 1H), 6.91 (d, J=9 Hz, 1H), 4.99 (br.s, 1H), 4.54-4.49 (m, 1H), 3.73 (s, 3H), 3.49-3.37 (m 2H), 3.19-3.14 (m, 2H), 2.94-2.89 (m, 2H), 1.73 (d, J=7 Hz, 3H).

### (R,S)-N-tertbutoxycarbonyl-5-methyl-2,3,4,5-tetrahydro-1H-benzo[d]azepin- 7-ol

To a flask containing (2-bromo-propyl)-[2-4-methoxy-phenyl)-ethyl]-amine hydrobromide (1.94 mmol, 0.68 g) is added aluminum chloride (5.81 mmol, 0.775 g). This is heated to 150° C. for 20 h. While the reaction is still warm, water (20 mL) is added, after 5 min EtOAc:CH₂Cl₂ (2:1, 50 mL) is added and the reaction allowed to cool to 20° C. with stirring. To this was added saturated NaHCO₃ (25 mL) to give an emulsion. The layers were separated. To the aqueous layer was added tetrahydrofuran (50 mL) and ditertbuyldicarbonate (1.9 mmol, 0.444 g). This was stirred for 20 h. The reaction was partitioned between EtOAc (100 mL) and saturated citric acid (100 mL). The layers were separated and the organics were dried with Na₂SO₄, filtered and concentrated to furnish 105 mg of white solid. ¹H NMR (400 Hz, MeOD) δ 6.89 (d, J=8 Hz, 1H), 6.59 (d, J=2 Hz, 1H), 6.51 (dd, J=6, 3 Hz, 1H), 3.72-3.60 (m, 2H), 3.41 (d, J=13 Hz, 1H), 3.25-3.15 (m, 1H), 3.00-2.92 (m, 2H), 2.74-2.71 (m, 1H), 1.42 (s, 9H), 1.25 (d, J=7 Hz, 3H). MS calculated for C₁₆H₂₃NO₃+H: 278, observed: 278.

### (R,S)-N-tertbutoxycarbonyl-8-benzyloxy-1-methyl-2,3,4,5-tetrahydro-1H-benzo[d]azepine

By the same general procedure as Example 8, (R,S)-N-tertbutoxycarbonyl-8-benzyloxy-1-methyl-2,3,4,5-tetrahydro-1H-benzo[d]azepine was obtained from N-tertbutoxycarbonyl-5-methyl-2,3,4,5-tetrahydro-1H-benzo[d]azepin-7-ol as an oil. MS calculated for C₂₃H₂₉NO₃+H: 368, observed: 368.

### (R,S)-8-benzyloxy-1-methyl-2,3,4,5-tetrahydro-1H-benzo[d]azepine

By the same general procedure as Example 1, (R,S)-8-benzyloxy-1-methyl-2,3,4,5-tetrahydro-1H-benzo[d]azepine was obtained from N-tertbutoxycarbonyl-8-benzyloxy-1-methyl-2,3,4,5-tetrahydro-1H-benzo[d]azepine hydrochloride as a white solid. MS calculated for C₁₈H₂₁NO+H: 268, observed: 268.

### Example 14

### (S)-7-Benzyloxy-1-methyl-2,3,4,5-tetrahydro-1H-benzo[d]azepine hydrochloride

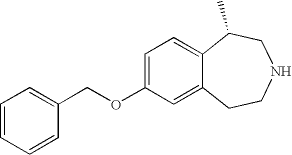

### (S)-N-trifluoroacetyl-1-methyl-2,3,4,5-tetrahydro-1H-benzo[d]azepin-7-ol

To a solution of (S)-N-trifluoroacetyl-7-methoxy-1-methyl-2,3,4,5-tetrahydro-1H-benzo[d]azepine (3.48 mmole, 1.0 g) in dichloromethane (25 mL) at 0° C. was added boron tribromide (1.0 M in dichloromethane, 8.0 mL). The reaction was stirred for 2 h and quenched with 1 M HCl (3 mL). This was extracted with dichlorometaane (2×10 mL). The organics were dried with MgSO₄, filtered and concentrated to furnish 900 mg of white solid. MS calculated for C₁₃H₁₄F₃NO₂+H: 274, observed: 274.

### (S)-N-trifluoroacetyl-7-benzyloxy-1-methyl-2,3,4,5-tetrahydro-1H-benzo[d]azepine

To a solution of (S)-N-trifluoroacetyl-1-methyl-2,3,4,5-tetrahydro-1H-benzo[d]azepin-7-ol (2.07 mmol, 0.568 g) in dichloromethane (10 mL) was added N′″-tertbutyl-N,N,N′,N′,N″, N″-hexamethylphosphorimidic triamide (2.49 mmol, 0.584 g), and benzyl bromide (4.15 mmol, 0.711 g). The reaction was stirred for 1 h, diluted with water (10 mL) and extracted with dichloromethane (2×10 mL). The organics were dried with MgSO₄, filtered and concentrated. The crude was purified by flash chromatography (10 to 25% EtOAc/hexanes) to furnish 0.616 g of a clear oil. ¹H NMR (400 MHz, DMSO) δ 7.41-7.25 (m, 5H), 7.17 (d, J=6 Hz, 1H), 6.88-6.86 (m, 2H), 5.06 (s, 2H), 3.41-3.32 (m, 4H), 3.18-2.96 (m, 3H), 1.43 (d, J=8 Hz, 3H). MS calculated for C₂₀H₂₀F₃NO ₂+H: 364, observed: 364.

### (S)-7-Benzyloxy-1-methyl-2,3,4,5-tetrahydro-1H-benzo[d]azepine hydrochloride

By the same general procedure as Example A, (S)-7-benzyloxy-1-methyl-2,3,4,5-tetrahydro-1H-benzo[d]azepine hydrochloride was obtained from (S)-N-trifluoroacetyl-7-benzyloxy-1-methyl-2,3,4,5-tetrahydro-1H-benzo[d]azepine as a white solid via hydrolysis and conversion to HCl salt. ¹H NMR (400 MHz, MeOD) δ 7.41-7.25 (m, 5H), 7.17 (d, J=6 Hz, 1H), 6.88-6.86 (m, 2H), 5.06 (s, 2H), 3.41-3.32 (m, 4H), 3.18-2.96 (m, 3H), 1.43 (d, J=8 Hz, 3H). MS calculated for C₁₈H₂₁NO+H: 268, observed: 268.

### Example 15

### (R)-1-Methyl-8-phenyl-2,3,4,5-tetrahydro-1H-benzo[d]azepine hydrochloride

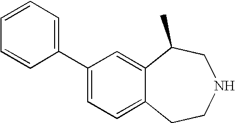

### (R)-N-tertbutoxycarbonyl-1-methyl-8-phenyl-1,2,4,5-tetrahydro-bentzo[d]azepine

A solution of (R)-N-tertbutoxycarbonyl-8-chloro-:1-methyl-2,3,4,5-tetrahydro-1H-benzo[d]azepine (0.68 mmol, 0.20 g) in tetrahydrofuran (5 mL) was treated with phenyl boronic acid (1.36 mmnol, 0.166 g), potassium phosphate (2.04 mmol, 0.432 g), palladium acetate (0.014 mmole, 0.003 g), 2-(dicyclohexylphosphino)-2′,4′,6′-tri-i-propyl-1,1′-biphenyl (0.034 mmole, 0.016 g) and heated to 120° C. for 30 m by microwave. The reaction was filtered through a celite and silica washing with ethyl acetate (10 mL) and concentrated. The crude material was purified by HPLC, which after concentration provided 247 mg of (R)-N-tertbutoxycarbonyl-8-1-Methyl-8-phenyl-1,2,4,5-tetrahydro-benzo[d]azepine. MS calculated for C₂₂H₂₇NO₂+H: 338, observed: 338.

### (R)-1-methyl-8-phenyl-2,3,4,5-tetrahydro-1H-benzo[d]azepine hydrochloride

To a solution of (R)-N-tertbutoxycarbonyl-1-methyl-8-phenyl-1,2,4,5-tetrahydro-benzo[d]azepine (0.71 mmole, 0.247 g) in methylene chloride (1 mL) was added 2.0 M HCl in diethyl ether (2.5 ml). This was stirred for 16 h, the liquid was decanted and the precipitate was washed with diethyl ether (2×2 mL) to give 120 mg of (R)-1-methyl-8-phenyl-2,3,4,5-tetrahydro-1H-benzo[d]azepine as an off white solid. ¹H NMR (400 MHz, DMSO-d₆) δ 7.60 (d, J=8 Hz, 2H), 7.42 (m, 4H), 7.33 (dd, J=8, 8 Hz, 1H) 7.25 (d, J=8 Hz, 1H), 3.45 (m, 1H), 3.36 (m, 1H), 3.28-3.16 (m, 3H), 3.03 (dd, J=7, 16 Hz, 1H), 2.95 (m, 2H), 1.39 (d, J=8 Hz, 3H). MS calculated for C₁₇H₁₉N+H: 238, observed: 238.

### Example 16

### (R,S)-7-Methoxy-1-methyl-8-phenyl-2,3,4,5-tetrahydro-1H-benzo [d]azepine hydrochloride

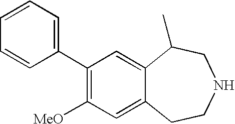

### N-trifluoroacetyl-7-Methoxy-1-methyl-8-phenyl-2,3,4,5-tetrahydro-1H-benzo[d]azepine

By the same general procedure as Example 15, N-trifluoroacetyl-7-Methoxy-1-methyl-8-phenyl-2,3,4,5-tetrahydro-1H-benzo[d]azepine was obtained from N-trifluoroacetyl-8-bromo-7-methoxy-1-methyl-2,3,4,5-tetrahydro-1H-benzo[d]azepine as an oil. MS calculated for C₂₀H₂₀F₃NO₂+H: 364, observed: 364.

### 7-Methoxy-1-methyl-8-phenyl-2,3,4,5-tetrahydro-1H-benzo[d]azepine hydrochloride

By the same general procedure as Example A, 7-Methoxy-1-methyl-8-phenyl-2,3,4,5-tetrahydro-1H-benzo[d]azepine was obtained from N-trifluoroacetyl-8-bromo-7-methoxy-1-methyl-2,3,4,5-tetrahydro-1H-benzo[d]azepine as an off-white solid via hydrolysis and conversion to HCl salt. MS calculated for C₁₈H₂₁NO+H: 268, observed: 268.

### Example 17

### (S)-8-(2-Fluoro-phenyl)-1-methyl-2,3,4,5-tetrahydro-1H-benzo[d]azepine hydrochloride

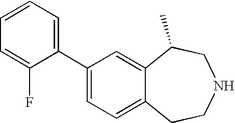

By the same general procedure as Example 15, (S)-8-2-fluoro-phenyl)-1-methyl-2,3,4,5-tetrahydro-1H-benzo[d]azepine hydrochloride was obtained from 2-fluorophenyl boronic acid as an off-white solid. MS calculated for C₁₇H₁₈FN+H: 256, observed: 256.

### Example 18

### (S)-8-(3-Fluoro-phenyl)-1-methyl-2,3,4,5-tetrahydro-1H-benzo[d]azepine hydrochloride

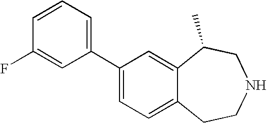

By the same general procedure as Example 15, (S)-8-(3-fluoro-phenyl)-1-methyl-2,3,4,5-tetrahydro-1H-benzo[d]azepine hydrochloride was obtained from 3-fluorophenyl boronic acid as an off-white solid. MS calculated for C₁₇H,₈FN+H: 256, observed: 256.

### Example 19

### (S)-8-(4-Fluoro-phenyl)-1-methyl-2,3,4,5-tetrahydro-1H-benzo[d]azepine hydrochloride

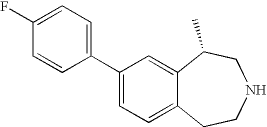

By the same general procedure as Example 15, (S)-8-(4-fluoro-phenyl)-1-methyl-2,3,4,5-tetrahydro-1H-benzo[d]azepine hydrochloride was obtained from 4-fluorophenyl boronic acid as an off-white solid. MS calculated for C₁₇H₁₈FN+H: 256, observed: 256.

### Example 20

### (S)-8-(2,6-Difluoro-phenyl)-1-methyl-2,3,4,5-tetrahydro-1H-benzo [d]azepine hydrochloride

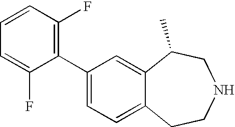

By the same general procedure as Example 15, (S)-8-(2,6difuoro-phenyl)-1-methyl-2,3,4,5-tetrahydro-1H-benzo[d]azepine hydrochloride was obtained from 2,6-difluorophenyl boronic acid as an off-white solid. MS calculated for C₁₇H₁₇F₂N+H: 274, observed: 274.

### Example 21

### (R)-8-(3-Fluoro-phenyl)-1-methyl-2,3,4,5-tetrahydro-1H-benzo[d]azepine hydrochloride

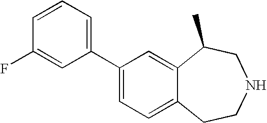

By the same general procedure as Example 15, (R)-8-(4-fluoro-phenyl)-1-methyl-2,3,4,5-tetrahydro-1H-benzo[d]azepine hydrochloride was obtained from 3-fluorophenyl boronic acid as an off-white solid. MS calculated for C₁₇H₁₈FN+H: 256, observed: 256.

### Example 22

### (R)-8-(4-Fluoro-phenyl)-1-methyl-2,3,4,5-tetrahydro-1H-benzo[d]azepine hydrochloride

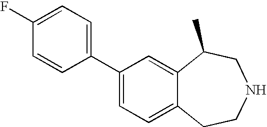

By the same general procedure as Example 15, (R)-8-(4-fluoro-phenyl)-1-methyl-2,3,4,5-tetrahydro-1H-benzo[d]azepine hydrochloride was obtained from 4-fluorophenyl boronic acid as an off-white solid. MS calculated for C₁₇H₁₈FN+H: 256, observed: 256.

### Example 23

### (R)-8-(2,3-Difluoro-phenyl)-1-methyl-2,3,4,5-tetrahydro-1H-benzo[d]azepine hydrochloride

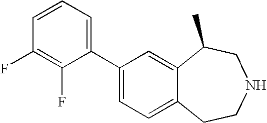

By the same general procedure as Example 15, (R)-8-(2,3-difluoro-phenyl)-1-methyl-2,3,4,5-tetrahydro-1H-benzo[d]azepine hydrochloride was obtained from 2,3difluorophenyl boronic acid as an off-white solid. MS calculated for C₁₇H₁₇F₂N+H: 274, observed: 274.

### Example 24

### (S)-8-(2,5-Difluoro-phenyl)-1-methyl-2,3,4,5-tetrahydro-1H-benzo[d]azepine hydrochloride

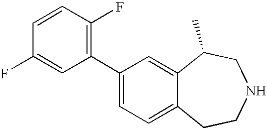

By the same general procedure as Example 15, (S)-8-(2,5-difluoro-phenyl)-1-methyl-2,3,4,5-tetrahydro-1H-benzo[d]azepine hydrochloride was obtained from 2,5-difluorophenyl boronic acid as an off-white solid. MS calculated for C₁₇H₁₇F₂N+H: 274, observed: 274.

### Example 25

### (R)-1-Methyl-8-pyridin-3-yl-2,3,4,5-tetrahydro-1H-benzo[d]azepine

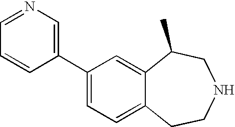

By the same general procedure as Example 15, (R)-1-methyl-8-pyridin-3-yl-2,3,4,5-tetrahydro-1H-benzo[d]azepine hydrochloride was obtained from 3-pyridyl boronic acid as an off-white solid. MS calculated for C₁₆H₁₈N₂+H: 239, observed: 239.

### Example 26

### (R,S)-1-Methyl-8-pyridin-2-yl-2,3,4,5-tetrahydro-1H-benzo[d]azepine

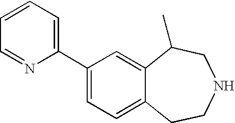

### (R,S)-trifluoro-methanesulfonic acid N-tertbutoxycarbony-5-methyl-2,3,4,5-tetrahydro-1H-benzo[d]azepin-8-yl ester

To a solution of (R,S)-N-tertbutoxycarbonyl-5-methyl-2,3,4,5-tetrrhydro-1H-benzo[d]azepin-8-ol (3.43 mmole, 951 mg) in dichloromethane (30 mL) was added pyridine (10.29 mmole, 1.15 mL) and trifluoromethanesulfonic anhydride (6.86 mmole, 0.83 mL). This was stirred for 20 h at 2 h. The reaction was concentrated and diluted with EtOAc (25 mL) and saturated NaHCO₃ (25 mL) added. The organics were washed with brine (10 mL), dried with Na₂SO₄, filtered and concentrated to afford 1.37 g of a clear oil. MS calculated for C₁₇H₂₂F₃NO₅S+H: 410, observed: 410.

### (R,S)-N-tertbutoxycarbonyl-1-methyl-8-pyridin-2-yl-2,3,4,5-tetrahydro-1H-benzo[d]azepine

A solution of (R,S)-trifluoro-methanesulfonic acid N-tertbutoxycarbony-5-methyl-2,3,4,5-tetrahydro-1H-benzo[d]azepin-7-yl ester (0.49 mmol, 0.200 g) in N-methyl-pyrrolidine (2 mL) was treated with bis(tri-t-butylphospine) palladium (0.024 mmol, 0.012 g), 2-pyridyliinc bromide (0.5 M in tetrahydrofuran, 2.0 mL) and heated to 150 C. for 30 m by microwave. The reaction was quenched with water (5 mL), and extracted with ethyl acetate (2×10 mL). The organics were combined, dried with MgSO₄, filtered and concentrated to furnish 35 mg of a clear oil. MS calculated for C₂₁H₂₆N₂O₂+H: 339, observed: 339.

### (R,S)-1-methyl-8-pyridin-2-yl-2,3,4,5-tetrahydro-1H-benzo[d]azepine hydrochloride

By the same general procedure as Example 15, (R,S)-1-methyl-8-pyridin-2-yl-2,3,4,5-tetrahydro-1H-benzo[d]azepine hydrochloride was obtained from N-tertbutoxycarbonyl-1-methyl-8-pyridin-2-yl-2,3,4,5-tetrahydro-1H-benzo[d]azepine as a white solid. MS calculated for C₁₆H₁₈N₂+H: 239, observed: 239.

### Example 27

Intracellular IP₃ Accumulation Assay

HEK293 cells were transfected in 15 cm sterile dishes with or without (control) 16 ug of human 5HT_{2C} receptor cDNA [Saltzman, A. G., et al. Biochem. Biophys. Res. Commun. 181, 1469-1478 (1991)] using 25 ul of lipofectamine. Cells were then incubated for 34 hours at 37° C./5% CO₂ and then transfection media was removed and replaced with 100 μL of DMEM. Cells were then plated onto 100 cm sterile dishes. The next day cells were plated into 96 well PDL microtiter plates at a density of 55K/0.2 ml. Six hours latter, media was exchanged with [³H]inositol (0.25 μCi/well) in inositol free DMEM and plates were incubated at 37° C./5% CO₂ overnight. The next day, wells were aspirated and 200 μL of DMEM containing test compound, 10 μM pargyline, and 10 mM LiCl was added to appropriate wells. Plates were then incubated at 37° C./5% CO₂ for three hours followed aspiration and by addition of fresh ice cold stop solution (1 M KOH, 19 mM Na-borate, 3.8 mM EDTA) to each well. Plates were kept on ice for 5-10 min and the wells were neutralized by addition of 200 μL of fresh ice cold neutralization solution (7.5% HCl). Plates were then frozen until further processing is desired. The lysate was then transferred into 1.5 mL Eppendorf tubes and 1 mL of chloroform/methanol (1:2) was added/tube. The solution was vortexed for 15 seconds and the upper phase was applied to a Biorad AG1-X8™ anion exchange resin (100-200 mesh). First, the resin was washed with water at 1:1.25 WN and 0.9 ml of upper phase was loaded onto the column. The column was then washed with 10 ml of 5 mM myo-inositol and 10 ml of 5 mM Na-borate/60 mM Na-formate. The inositol tris phosphates were eluted into scintillation vials containing 10 ml of scintillation cocktail with 2 ml of 0.1 M formic acid/1 M ammonium formate. The columns were regenerated by washing with 10 mL of 0.1 M formic acid/3 M ammonium formate and rinsed twice-with dd H₂O and stored at 4° C. in water.

### Example 28

Ihibition of Food Intake in Food-Deprived Rats

Male Sprague-Dawley rats (250-350 g) are deprived of food overnight prior to testing. Prior to food deprivation, the animals are weighed and separated into treatment groups in order to balance groups according to body weight. On the test day, animnals are placed into individual cages (no bedding) at 9:00am with free access to water. At 10:00 AM, animals are injected with test compound (p.o., i.p., or s.c.) and then presented with a pre-weighed amount of food in a dish either 60 min (p.o.) or 30 min (i.p. and s.c.) after drug administration. Food consumption over different time points is determined by weighing the food cup at 1, 2, 4, and 6 hr after the food is presented. Thus, food consumption is measured at 2, 3, 5, and 7 hr post-injection in p.o. studies, and at 1.5, 2.5, 4.5, and 6.5 hr post-injection in i.p. and s.c. studies.

### Example 29

Compound Activity

Compounds of Examples 1-12 were tested for 5HT_{2C} agonist activity according to the assay of Example 27. All of these compounds were found to be active and have an IC₅₀ value between about 1 nM and about 1.3 μM. Compounds of Examples 13-26 were tested for 5HT_{2C} agonist activity according to the assay of Example 27. The majority of these compounds were showed to be active and have an IC₅₀ value of less than 10 μM.

Each embodiment of the present may in the alternative be limited to relate to those compounds that have about 2 fold or greater 5HT_{2C} agonist activity compared to 5HT_{2B} agonist activity. In some embodiments, compounds of present invention have about 3, 4, 5, 6, 7, 8, 9, 10 fold or greater 5HT_{2C} agonist activity compared to 5HT_{2B} agonist activity.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A compound of Formula (I): 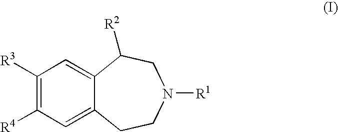
or pharmaceutically acceptable salt thereof, wherein:
R¹ is H or C₁-C₈ alkyl;
R² is C₁-C₈ alkyl;
R³ is H, aryl, arylalkyl-O—, arylalkyl-N(R⁵)—, aryl-N(R⁵)—, or heteroaryl, wherein said aryl is optionally substituted with up to two substituents selected from C₁₋₈ alkyl, halogen, perhaloalkyl, and alkoxy;
R⁴ is H, arylalkyl-O—, alkoxy, or aryloxy; and
R⁵ is H, C₁-C₈ alkyl, aryl, C₁₋₈ alkenyl, heteroaryl, arylalkyl, heteroarylalkyl, perhaloalkyl, or allyl;
with the provisos:
a) at least one of R³ and R⁴ is other than H;
b) when R³ is arylalkyl-N(R⁵)— or aryl-N(R⁵)— and R⁴ is H, then R¹ is H;
c) when R¹ is H, R² is CH₃ and R³ is 2-chlorophenyl, then R⁴ is other than H; and
d) when R¹ is H, R² is CH₃ and R³ is 2-thienyl, then R⁴ is other than methoxy.

2. The compound of claim 1 wherein:
R¹ is H or C₁-C₈ alkyl;
R² is C₁-C₈ alkyl;
R³ is H, aryl, arylalkyl-O—, arylalkyl-N(R⁵)—, or aryl-N(R⁵)— wherein said aryl is optionally substituted with up to two substituents selected from C₁₋₈ alkyl, halogen, perhaloalkyl, and alkoxy;
R⁴ is H or aryloxy; and
R⁵ is H, C₁-C₈ alkyl, aryl, C₁₋₈ alkenyl, heteroaryl, arylalkyl, heteroarylalkyl, perhaloalkyl, or allyl.

3. A compound of claim 1 having Formula (Ia): 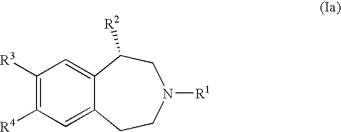
or pharmaceutically acceptable salt form thereof.

4. A compound of claim 1 having Formula (Ib): 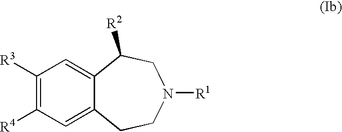
or pharmaceutically acceptable salt form thereof.

5. The compound of claim 1 wherein:
R¹ is H or C₁-C₈ alkyl;
R² is C₁-C₈ alkyl;
R³ is arylalkyl-O—, arylalkyl-N(R⁵)—, or aryl-N(R⁵)—;
R⁴ is H; and
R⁵ is H, C₁-C₈ alkyl, aryl, C₁₋₈ alkenyl, heteroaryl, arylalkyl, heteroarylalkyl, perhaloalkyl, or allyl.

6. The compound of claim 1 wherein:
R¹ is H or C₁-C₈ alkyl;
R² is C₁-C₈ alkyl;
R³ is arylalkyl-O—, arylalkyl-N(R⁵)—, or aryl-N(R⁵)—;
R⁴ is H; and
R⁵ is H, C₁-C₈ alkyl, or aryl.

7. The compound of claim 1 wherein:
R¹ is H or C₁-C₈ alkyl;
R² is C₁-C₈ alkyl;
R³ is arylalkyl-O—; and
R⁴ is H.

8. The compound of claim 1 wherein:
R¹ is H or C₁-C₈ alkyl;
R² is C₁-C₈ alkyl;
R³ is H; and
R⁴ is arylalkyl-O—.

9. The compound of claim 1 wherein:
R¹ is H;
R² is C₁-C₄ alkyl;
R³ is arylalkyl-O—; and
R⁴ is H.

10. The compound of claim 1 wherein:
R¹ is H;
R² is C₁-C₈ alkyl;
R³ is arylalkyl-N(R⁵)—;
R⁴ is H; and
R⁵ is H, C₁-C₈ alkyl, or aryl.

11. The compound of claim 1 wherein:
R¹ is H;
R² is methyl;
R³ is phenyl, phenylalkyl-O—, phenylalkyl-N(R⁵)—, or phenyl-N(R⁵)—;
R⁴ is H; and
R⁵ is H.

12. The compound of claim 1 wherein:
R¹ is H;
R² is methyl;
R³ is H; and
R⁴ is phenylalkyl-O—.

13. The compound of claim 1 wherein:
R¹ is H;
R² is methyl;
R³ is phenyl optionally substituted with up to two halogens, or R³ is pyridinyl; and
R⁴ is H or alkoxy.

14. The compound of claim 1 wherein:
R¹ is H;
R² is methyl;
R³ is phenyl optionally substituted with up to two fluoro; and
R⁴ is H or methoxy.

15. The compound of claim 1 wherein:
R¹ is H;
R² is methyl;
R³ is pyridinyl; and
R⁴ is H.

16. A compound of any one of claims 1 to 4 selected from:
a) 7-benzyloxy-1-methyl-2,3,4,5-tetrahydro-1H-benzo[d]azepine;
b) 1-methyl-7-(1-phenyl-ethoxy)-2,3,4,5-tetrahydro-1H-benzo[d]azepine;
c) 1-methyl-7-phenethyloxy-2,3,4,5-tetrahydro-1H-benzo[d]azepine;
d) 1-methyl-7-(3-phenyl-propoxy)-2,3,4,5-tetrahydro-1H-benzo[d]azepine;
e) benzyl-(5-methyl-2,3,4,5-tetrahydro-1H-benzo[d]azepin-7-yl)-amine;
f) (5-methyl-2,3,4,5-tetrahydro-1H-benzo[d]azepin-7-yl)-(1′-phenyl-ethyl)-amine;
g) benzyl-methyl-(5-methyl-2,3,4,5-tetrahydro-1H-benzo[d]azepin-7-yl)-amine;
h) (5-methyl-2,3,4,5-tetrahydro-1H-benzo[d]azepin-7-yl)-phenethyl-amine;
i) (5-methyl-2,3,4,5-tetrahydro-1H-benzo[d]azepin-7-yl)-(3-phenyl-propyl)-amine;
j) (5-methyl-2,3,4,5-tetrahydro-1H-benzo[d]azepin-7-yl)-phenyl-amine; and
k) 1-methyl-8-phenyl-2,3,4,5-tetrahydro-1H-benzo[d]azepine;
or pharmaceutically acceptable salt thereof.

17. A compound of any one of claims 1 to 4 selected from:
m) 8-Benzyloxy-1-methyl-2,3,4,5-tetrahydro-1H-benzo[d]azepine;
n) 7-Benzyloxy-1-methyl-2,3,4,5-tetrahydro-1H-benzo[d]azepine;
o) 1-Methyl-8-phenyl-2,3,4,5-tetrahydro-1H-benzo[d]azepine;
p) 7-Methoxy-1-methyl-8-phenyl-2,3,4,5-tetrahydro-1H-benzo[d]azepine;
q) 8-(2-Fluoro-phenyl)-1-methyl-2,3,4,5-tetrahydro-1H-benzo[d]azepine;
r) 8-(3-Fluoro-phenyl)-1-methyl-2,3,4,5-tetrahydro-1H-benzo[d]azepine;
s) 8-(4-Fluoro-phenyl)-1-methyl-2,3,4,5-tetrahydro-1H-benzo[d]azepine;
t) 8-(2,6-Difluoro-phenyl)-1-methyl-2,3,4,5-tetrahydro-1H-benzo[d]azepine;
u) 8-(2,3-Difluoro-phenyl)-1-methyl-2,3,4,5-tetrahydro-1H-benzo[d]azepine;
v) 8-(2,5-Difluoro-phenyl)-1-methyl-2,3,4,5-tetrahydro-1H-benzo[d]azepine;
w) 1-Methyl-8-pyridin-3-yl-2,3,4,5-tetrahydro-1H-benzo[d]azepine; and
x) 1-Methyl-8-pyridin-2-yl-2,3,4,5-tetrahydro-1H-benzo[d]azepine;
or pharmaceutically acceptable salt thereof.

18. A composition comprising a compound of claim 1 and a pharmaceutically acceptable carrier.

19. A method of treating disorders of the central nervous system, damage to the central nervous system, cardiovascular disorders, gastrointestinal disorders, diabetes insipidus, sleep apnea or HDL-related condition comprising administering to a patient in need of said treating a therapeutically effective amount of a compound of claim 1.

20. The method of claim 19 wherein the disorders of the central nervous system are selected from depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension.

21. The method according to claim 19 wherein the disorder of the central nervous system is obesity.

22. The method according to claim 19 wherein the sexual dysfunction is male erectile dysfunction.

23. A method of decreasing food intake of a mammal comprising administering to said mammal a therapeutically effective amount of a compound of claim 1.

24. A method of inducing satiety in a mammal comprising administering to said mammal a therapeutically effective amount of a compound of claim 1.

25. A method of controlling weight gain of a mammal comprising administering to said mammal a therapeutically effective amount of a compound of claim 1.

26. A method of treating obesity comprising administering to a patient in need of such treating a therapeutically effective amount of a compound of claim 1.

27-41. (canceled)

42. A method for preparing a pharmaceutical composition comprising the step of mixing a compounds of claim 1 and a pharmaceutically acceptable carrier.
